SEQUENCE LISTING (12 sequences)

SEQ ID NO: 1 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 2 - DNA, 792 bp, Homo sapiens
atgatcgagacatacaaccagacaagccctagaagcgccgccacaggactgcctatcagcatgaagatcttcatgtacctgctgaccgtgttcctgatcacccagatgatcggcagcgccctgtttgccgtgtacctgcacagacggctggacaagatcgaggacgagagaaacctgcacgaggacttcgtgttcatgaagaccatccagcggtgcaacaccggcgagagaagtctgagcctgctgaactgcgaggaaatcaagagccagttcgagggcttcgtgaaggacatcatgctgaacaaagaggaaacgaagaaagagaactccttcgagatgcagaagggcgaccagaatcctcagatcgccgctcacgtgatcagcgaggccagcagcaagacaacaagcgtgctgcagtgggccgagaagggctactacaccatgagcaacaacctggtcaccctggagaacggcaagcagctgacagtgaagcggcagggcctgtactacatctacgcccaagtgaccttctgcagcaacagagaggccagctctcaggctcctttcatcgccagcctgtgcctgaagtctcctggcagattcgagcggattctgctgagagccgccaacacacacagcagcgccaaaccttgtggccagcagtctattcacctcggcggagtgtttgagctgcagcctggcgcaagcgtgttcgtgaatgtgacagaccctagccaggtgtcccacggcaccggctttacatctttcggactgctgaagctgtgatgatag

SEQ ID NO: 3 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 4 - DNA, 768 bp, Homo sapiens
atggaatacgccagcgacgcctctctggaccctgaagctccttggcctccagctcctagagccagggcttgtagagtgctgccttgggctcttgtggctggacttctgcttctgttgctcctggctgctgcctgcgcagtgtttcttgcttgtccatgggctgtgtcaggagccagagcatctcctggatctgccgcttctcccagactgagagagggacctgaactgagccctgatgatcctgctggactgctcgacctgagacagggcatgtttgcccagctggtggcccagaatgtgctgctgattgatggccctctgagctggtacagcgatcctggacttgctggcgttagcctgactggaggcctgagctacaaggaggacaccaaagaactggtggtggccaaggctggcgtgtactacgtgttctttcagctggaactgcggagagtggtggcaggcgaaggatctggatccgtgtctctggcactgcatctgcagcctctgagatctgctgctggtgcagctgccctggctctgacagttgatctgcctcctgcctccagcgaagccagaaacagcgcctttggcttccaaggcagactgctgcacctgtctgctggccagagactgggagtgcacctccacacagaagcaagagcaagacacgcctggcagcttacacaaggcgctacagtgctgggcctgttcagagtgacacctgagattccagctggcttgccatctcctcgcagcgagtaatga

SEQ ID NO: 5 - protein, 666 aa, Artificial sequence
  Synthetic ERV-K-gag sequence
MGQTKSKIKSKYASYLSFIKILLKRGGVKVSTKNLIKLFQIIEQFCPWFPEQGTLDLKDWKRIGKELKQAGRKGNIIPLTVWNDWAIIKAALEPFQTEEDSVSVSDAPGSCIIDCNENTRKKSQKETESLHCEYVAEPVMAQSTQNVDYNQLQEVIYPETLKLEGKGPELVGPSESKPRGTSPLPAGQVPVTLQPQKQVKENKTQPPVAYQYWPPAELQYRPPPESQYGYPGMPPAPQGRAPYPQPPTRRLNPTAPPSRQGSELHEIIDKSRKEGDTEAWQFPVTLEPMPPGEGAQEGEPPTVEARYKSFSIKMLKDMKEGVKQYGPNSPYMRTLLDSIAHGHRLIPYDWEILAKSSLSPSQFLQFKTWWIDGVQEQVRRNRAANPPVNIDADQLLGIGQNWSTISQQALMQNEAIEQVRAICLRAWEKIQDPGSTCPSFNTVRQGSKEPYPDFVARLQDVAQKSIADEKARKVIVELMAYENANPECQSAIKPLKGKVPAGSDVISEYVKACDGIGGAMHKAMLMAQAITGVVLGGQVRTFGGKCYNCGQIGHLKKNCPVLNKQNITIQATTTGREPPDLCPRCKKGKHWASQCRSKFDKNGQPLSGNEQRGQPQAPQQTGAFPIQPFVPQGFQGQQPPLSQVFQGISQLPQYNNCPPPQAAVQQ

SEQ ID NO: 6 - DNA, 2004 bp, Artificial sequence
  Synthetic ERV-K-gag sequence
atgggacagaccaagagtaagatcaagtctaagtacgccagctacctcagcttcatcaagatcctgctgaagagaggaggcgtgaaagtgtccaccaagaacctgatcaagctgttccagatcatcgagcagttctgtccctggtttcctgagcagggcaccctggatctgaaggactggaagcggatcggcaaagagctgaagcaggctggcagaaagggcaacatcatccctctgaccgtgtggaacgactgggccatcatcaaagcagctctggaacccttccagaccgaagaggatagcgtgtccgtgtctgatgctcctggcagctgcatcatcgactgcaacgagaacacccggaagaagtcccagaaagagacagagagcctgcactgcgagtacgtggccgaacctgtgatggctcagagcacccagaacgtggactacaaccagctccaagaagtgatctatcccgaaacactgaagctggaaggcaagggacctgaactcgtgggtccttctgagtctaagcccagaggcacatctcctctgcctgcaggacaggtgccagtgacactgcagcctcagaaacaagtgaaagagaacaagacccagcctcctgtggcctaccagtattggcctccagccgagctgcagtacagacctcctccagagagccagtacggctaccctggaatgcctcctgctcctcaaggcagagctccttatcctcagcctcctaccagacggctgaaccctacagctcctcctagcagacagggctctgagctgcacgagatcattgacaagagccggaaagagggcgacaccgaggcttggcagtttcccgttacactggaacccatgcctccaggcgaaggcgctcaagaaggcgaacctcctacagtggaagccaggtacaagagcttcagcatcaagatgctgaaggacatgaaggaaggcgtcaagcagtacggacctaacagcccatacatgcggaccctgctggattctattgcccacggccaccggctgatcccttacgattgggagatcctggctaagtcctctctgagccctagccagttcctgcagttcaagacctggtggatcgacggcgtgcaagaacaagtgagacggaacagagctgccaatcctcctgtgaacatcgacgccgaccagctcctcggaatcggccagaattggagcaccatctctcagcaggctctgatgcagaacgaggccattgaacaagtcagagccatctgcctgagagcttgggagaagattcaggacccaggcagcacatgtcccagcttcaataccgttcggcagggcagcaaagagccctatcctgactttgtggctagactgcaggatgtggcccagaagtctattgccgacgagaaggctcggaaagtgatcgtggaactgatggcctacgagaacgctaatccagagtgccagagcgccatcaagcccttgaagggcaaagtgcctgccggatccgatgtgatcagcgagtatgtgaaggcctgcgacggaatcggaggtgccatgcacaaagccatgctgatggcacaggccatcactggcgttgtgctcggaggacaagttcggacctttggaggcaagtgctacaactgtggccagatcggacacctgaagaagaactgccctgtgctgaacaagcagaacatcaccatccaggccaccaccaccggcagagaacctccagatctgtgccctagatgcaagaagggcaagcactgggccagccagtgcagaagcaagttcgacaagaacggccagcctctgagcggcaacgaacaaagaggacagcctcaggctcctcagcagactggcgcatttccaatccagcccttcgtgcctcaaggcttccagggacaacagcctccactgtctcaggtgttccagggcattagccagctccctcagtacaacaactgccctccacctcaggctgctgtgcagcagtgatga

SEQ ID NO: 7 - protein, 699 aa, Artificial sequence
  Synthetic ERV-K-env/MEL sequence
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPMIRAVTWMDNPIEVYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKMLAVISCAVQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 8 - DNA, 2103 bp, Artificial sequence
  Synthetic ERV-K-env/MEL sequence
atgaaccctagcgagatgcagagaaaggctccacctagacggagaagacacagaaacagggctcctctgacacacaagatgaacaagatggtcaccagcgaggaacagatgaaactgcccagcaccaagaaggccgagcctccaacatgggctcagctgaagaaactgacccagctggccaccaagtacctggagaacaccaaagtgacccagacacctgagagcatgctgctggcagctctgatgatcgtgtccatggtggtgtccctgcctatgcctgctggtgctgccgctgccaactacacatactgggcctacgtgccctttcctcctatgatcagagccgtgacctggatggacaaccctattgaggtgtacgtgaacgacagcgtgtgggtgccaggacctatcgacgatagatgtcctgccaaacctgaggaagagggcatgatgatcaacatcagcatcggctaccggtatcctccaatctgcctgggcagagcacctggctgtcttatgccagctgtgcagaattggctggtggaagtgcctaccgtgtctcccatcagccggttcacctaccacatggtgtccggcatgagcctcagacctagagtgaactacttgcaggacttcagctatcagcggagcctgaagttcagacccaagggaaagccctgtcctaaagagattcccaaagagtccaagaacaccgaggtgctcgtgtgggaagagtgcgtggccaattctgccgtgatcctgcagaacaacgagttcggcaccatcattgactgggctcctagaggccagttctaccacaattgcagcggacagacacagagctgtcctagcgcacaagtgtcaccagccgtggatagcgatctgaccgagagcctggacaagcacaaacacaagaaacttcagagcttctatccctgggagtggggagagaagggcatctctacaccaaggcctaagatcattagccctgtgtctggaccagaacatcccgaactttggagactgacagtggccagccaccacatcagaatctggagcggcaatcagaccctggaaacacgggacagaaagcccttctacaccgtcgatctgaacagcagcctgaccgtgcctctccagagctgtgtgaagcctccttacatgctggtcgtgggcaacattgtgatcaagcccgactcccagaccatcacatgcgagaactgcagactgctgacctgcatcgacagcaccttcaactggcagcaccggatcctgctcgtgcgagctagagaaggcgtgtggatccctgtctctatggacaggccttgggaagccagccctagcgtgcacattctgacagaggtgctgaagggcgtgctcaacagatccaagcggttcatcttcaccctgatcgccgtcatcatgggcctgattgctgtgacagccacagctgctgttgctggcgtggccctgcatagctctgtgcagagcgtgaacttcgtgaacgattggcagaagaacagcacacggctgtggaacagccagagcagcatcgaccagaagatgctggccgtgatctcctgtgccgtgcagacagttatctggatgggcgacagactgatgagcctggaacaccggttccagctgcagtgcgactggaataccagcgacttctgcatcacacctcagatctacaacgagagcgagcaccactgggatatggtccgaaggcatctgcagggcagagaggacaacctgacactggacatcagcaagctgaaagagcagatcttcgaggccagcaaggctcacctgaatctggtgcctggaaccgaagctattgctggagttgcagatggcctggccaatctgaatcctgtgacctgggtcaagaccatcggcagcaccacaatcatcaacctgatcctgatcctcgtgtgcctgttttgcctgctgcttgtgtgcagatgcacccagcagctgagaagagacagcgaccatagagaaagagccatgatgaccatggccgtcctgagcaagagaaagggaggcaacgtgggcaagagcaagcgggatcagatcgtgaccgtgtccgtttgataa

SEQ ID NO: 9 - protein, 741 aa, Artificial sequence
  Synthetic hFOLR1-hPRAME fusion sequence
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTTKAKVDGLSTEAEQPFIPVEVLVDLFLKEGACDELFSYLIEKVAAKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 10 - DNA, 2228 bp, Artificial sequence
  Synthetic hFOLR1-hPRAME fusion nucleotide sequence
tggcccagagaatgaccacacaactgctgctgctcctggtgtgggttgccgttgttggagaggcccagaccagaattgcctgggccagaaccgagctgctgaacgtgtgcatgaacgccaagcatcacaaagagaagcctggacctgaagacaagctgcatgaacagtgtcggccttggagaaagaatgcttgctgtagcaccaacaccagccaagaggcccacaaggacgtgtcctacctgtaccggttcaactggaaccactgcggagaaatggctcctgcctgcaagagacacttcatccaggatacctgcctgtacgagtgctctcccaatctcggaccttggatccagcaagtggaccagagctggcggaaagaacgggtgctgaatgtgcccttgtgcaaagaggattgcgagcagtggtgggaagattgccggaccagctacacatgtaagagcaactggcacaaaggctggaactggaccagcggcttcaacaagtgtgccgtgggagctgcctgccagcctttccacttctacttcccaacacctaccgtgctgtgcaacgaaatctggacccacagctacaaggtgtccaactacagcagaggcagcggcaggtgtatccagatgtggttcgatcccgctcagggcaatcccaatgaggaagtggctagattctacgctgctgccatggaaagaagaaggctctggggcagcatccagagccggtacattagcatgagcgtgtggacaagccctagacggctggttgaactggctggacagagcctgctcaaggatgaggccctggccattgctgctctggagctgctgcctagagagctgttccctcctctgttcatggctgccttcgacggcagacacagccagacactgaaagccatggtgcaggcctggcctttcacctgtctgcctctgggagtgctgatgaagggccagcatctgcacctggaaaccttcaaggccgtgctggacggcctggatgttctcctggctcaagaggtgaggcctcggcgttggaaactgcaggttctggatctgcggaagaactctcaccaggatttctggaccgtttggtccggcaacagagccagcctgtacagctttcctgaacctgaggctgcccagcccatgaccacaaaggccaaagtggatggcctgagcacagaggccgagcagcctttcattcccgtcgaagtgctggtggacctgttcctgaaagaaggagcctgcgatgagctgttcagctacctgattgagaaggtggcagccaagaagaacgtgctgcggctgtgctgcaagaagctgaagatctttgccatgcctatgcaggatatcaagatgatcctgaagatggtgcagctggacagcatcgaggacctggaagtgacctgtacctggaagctgcccacactggccaagttcagcccttacctgggacagatgattaacctgcggaggctgctgctgtctcacatccacgccagctcctacatcagccctgagaaagaggaacagtatatcgcccagttcacaagccagtttctgagcctgcagtgtctgcaggccctgtacgtggacagcctgttctttctgagaggcaggctggatcagctgctgcggcacgtgatgaaccctctggaaaccctgagcatcaccaactgtagactgagcgagggcgacgtgatgcacctgtctcagagcccatctgtgtctcagctgagcgtgctgtctctgtctggcgtgatgctgaccgatgtgagccctgaacctctgcaggcactgctggaaagagcctccgctactctgcaggacctggtgttcgatgagtgcggcatcaccgatgaccagctgcttgctctgctgccaagcctgagccactgtagccagctgacaaccctgtccttctacggcaacagcatctccatctctgccctgcagtctctcctgcagcatctgatcggcctgtccaatctgacccacgtgctgtaccctgtgccactggaaagctacgaggacatccacggaaccctgcacctcgagagactggcctatctgcatgctcggctgagagaactgctgtgcgaactgggcagacccagcatggtttggctgagcgccaatccatgtcctcactgtggcgaccggaccttctacgaccctgagcctatcctgtgtccttgcttcatgcccaactaatag

SEQ ID NO: 11 - protein, 517 aa, Artificial sequence
  Synthetic ERV-K-env/MEL_03 sequence
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPMIRAVTWMDNPIEVYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNMLAVISCAVAGVALHGSAGSAAGSGEFVVISAILALVVLTIISLIILIMLWQKKPR

SEQ ID NO: 12 - DNA, 1557 bp, Artificial sequence
  ERV-K-env/MEL_03 nucleotide sequence
atgaaccctagcgagatgcagagaaaggctccacctagacggagaagacacagaaacagggctcctctgacacacaagatgaacaagatggtcaccagcgaggaacagatgaaactgcccagcaccaagaaggccgagcctccaacatgggctcagctgaagaaactgacccagctggccaccaagtacctggagaacaccaaagtgacccagacacctgagagcatgctgctggcagctctgatgatcgtgtccatggtggtgtccctgcctatgcctgctggtgctgccgctgccaactacacatactgggcctacgtgccctttcctcctatgatcagagccgtgacctggatggacaaccctattgaggtgtacgtgaacgacagcgtgtgggtgccaggacctatcgacgatagatgtcctgccaaacctgaggaagagggcatgatgatcaacatcagcatcggctaccggtatcctccaatctgcctgggcagagcacctggctgtcttatgccagctgtgcagaattggctggtggaagtgcctaccgtgtctcccatcagccggttcacctaccacatggtgtccggcatgagcctcagacctagagtgaactacttgcaggacttcagctatcagcggagcctgaagttcagacccaagggaaagccctgtcctaaagagattcccaaagagtccaagaacaccgaggtgctcgtgtgggaagagtgcgtggccaattctgccgtgatcctgcagaacaacgagttcggcaccatcattgactgggctcctagaggccagttctaccacaattgcagcggacagacacagagctgtcctagcgcacaagtgtcaccagccgtggatagcgatctgaccgagagcctggacaagcacaaacacaagaaacttcagagcttctatccctgggagtggggagagaagggcatctctacaccaaggcctaagatcattagccctgtgtctggaccagaacatcccgaactttggagactgacagtggccagccaccacatcagaatctggagcggcaatcagaccctggaaacacgggacagaaagcccttctacaccgtcgatctgaacagcagcctgaccgtgcctctccagagctgtgtgaagcctccttacatgctggtcgtgggcaacattgtgatcaagcccgactcccagaccatcacatgcgagaactgcagactgctgacctgcatcgacagcaccttcaactggcagcaccggatcctgctcgtgcgagctagagaaggcgtgtggatccctgtctctatggacaggccttgggaagccagccctagcgtgcacattctgacagaggtgctgaagggcgtgctcaacatgctggccgtgatctcctgtgccgtggctggcgtggccctgcatggctctgctggatctgctgctggaagcggcgagttcgtggtcatctctgccattctggctctggtggtgctgaccatcatcagcctgatcatcctgattatgctgtggcagaagaagccccggtgataa

(19) United States
(12) Patent Application Publication
(10) Pub. No.: US 2023/0059344 A1
(43) Pub. Date: Feb. 23, 2023

(54) Medical Uses of 4-1BBL Adjuvanted Recombinant Modified Vaccinia Virus Ankara (MVA)

(71) Applicant: Bavarian Nordic A/S, Hellerup (DK)

(72) Inventors: Maria Hinterberger, Sauerlach (DE); José Medina Echeverz, Munich (DE); Mathias Habjan, Puchheim (DE); Jügen Hausmann, Gundelfingen (DE); Markus Kalla, Penzberg (DE)

(73) Assignee: Bavarian Nordic A/S, Hellerup (DK)

(21) Appl. No.: 17/778,398

(22) PCT Filed: Nov. 20, 2020
(86) PCT No.: PCT/EP2020/082888

§ 371 Date: May 19, 2022

(30) Foreign Application Priority Data
Nov. 20, 2019 (EP) 19210369.5
Aug. 14, 2020 (EP) 20191226.8
Aug. 31, 2020 (EP) 20193706.7

(51) Int. Cl. C12N 7/00; A61P 35/00; C12N 15/79; C07K 14/705

(52) U.S. Cl. CPC C12N 7/00; A61P 35/00; C12N 15/79; C07K 14/70575; C12N 2710/24143; C07K 2319/00

(57) ABSTRACT

The invention relates to a recombinant Modified Vaccinia Virus Ankara (MVA) expressing a TAA and the costimulatory molecule 4-1BBL for use in (i) the prevention of recurrence of a solid tumor, wherein the recombinant MVA is intratumorally administered to the solid tumor, or (ii) the treatment, prevention and/or prevention of recurrence of a tumor, wherein the recombinant MVA is intratumorally administered to another solid tumor.

### TECHNICAL FIELD

The present invention relates to the field of cancer immunotherapy, particularly to oncolytic virotherapy. More specifically, the invention relates to a recombinant Modified Vaccinia Virus Ankara (MVA) expressing a tumor associated antigen (TAA) and the costimulatory molecule 4-1BBL for use in the treatment, prevention and/or prevention of recurrence of tumors and metastases, wherein the recombinant MVA is intratumorally administered to a solid tumor. The invention also relates to respective methods of treatment.

### BACKGROUND

Human cancers are extraordinary heterogenous in terms of tumor antigen expression and immune infiltration and composition. A common feature, however, is the host inability to mount potent immune responses that prevent tumor growth effectively. Often, naturally primed CD8⁺ T cells against solid tumors lack adequate stimulation and efficient penetration of tumor tissue due to the hostile tumor microenvironment.

The lack of potent immune responses against solid tumors due to the poor capacity of immune cells to infiltrate or perform effector functions in the hostile tumor microenvironment (TME) is a major challenge for cancer immunotherapy [1]. The concept of reprogramming the immunosuppressive TME into an inflammatory one by tumor-directed therapy has attracted much attention in recent years [2]. The aim is to activate immune cells that have already homed to the tumor and local lymph nodes or recruit new immune cells to the TME, while minimizing irrelevant activation of the rest of the immune system [3]. To achieve this, several strategies are being explored in preclinical models as well as in the clinic, either employing the local release and activation of biochemical signals derived from pathogen recognition and unprogrammed cell destruction or local administration of immunostimulatory monoclonal antibodies and cytokines [2].

Local oncolytic virotherapy relies on the concept of tumor-targeted therapy through specific infection and destruction of tumor cells and modulation of the TME. The recent Food and Drug Administration (FDA) approval of the first-in-class oncolytic agent IMLYGIC, a modified herpes simplex virus 1 encoding human GM-CSF, for stage III melanoma patients [4], emphasized the great potential of oncolytic viruses (OV). There is a wide spectrum of viral families that have been investigated for their oncolytic effects, including herpesvirus, poxvirus and adenovirus, among others [5].

While historically tumor cell-specific replication and direct killing activity of OVs viruses were considered the primary mode of action, initiation or augmentation of a host anti-tumor immune response is now known to be essential for oncolytic virotherapy [6]. Hence, local virotherapy can be regarded as an in situ vaccine that leads to the release of damage- or pathogen-associated molecular patterns and immunogenic cell death accompanied by tumor antigen release which ultimately results in the initiation of innate and adaptive anti-tumor immune responses [7].

A salient feature of most OVs is their tumor specificity and their capacity to spread through tumor tissue by viral replication and concomitant tumor cell lysis [8]. Despite their engineered tissue tropism for tumor cells, the use of replication-competent viruses in patients still raises safety concerns. MVA-BN is a highly attenuated vaccinia strain approved by the FDA (JYNNEOS®) as a non-replicating vaccine against smallpox and monkeypox [9]. In addition, a recombinant MVA-BN vaccine vector has recently been approved by European Medicines Agency (EMA) as part of an Ebola vaccine and others are employed in clinical trials against various infectious agents [10]. MVA is a potent inducer of Type I interferons (IFN) [11, 12, 13] and elicits robust humoral and cellular immune responses against vector-encoded heterologous antigens [14, 16]. Importantly, MVA cannot replicate in human cells as its replication ability is restricted to embryonic avian cells [15]. Thus, the excellent safety profile and immune stimulatory properties of MVA make it a prime candidate for therapeutic interventions.

MVA can accommodate large transgene inserts facilitating the incorporation of heterologous antigens and immune stimulatory molecules to elicit antigen-specific T cell responses and enhance certain immune-activating pathways. CD40L-adjuvanted MVA drastically augmented innate and adaptive immune responses upon IV injection [16, 17]. Furthermore, OVs genetically altered with co-stimulatory molecules or inflammatory cytokines increased therapeutic efficacy after intratumoral (IT) therapy [18]. Hence, IT treatment with MVA encoding a tumor associated antigen (TAA) together with a co-stimulatory molecule might enhance anti-tumor immune responses in the TME.

The tumor necrosis factor receptor (TNFR)-family member 4-1BB or CD137 is defined as a bona fide co-stimulatory molecule in T cells. 4-1BB is transiently induced upon TCR stimulation and subsequent engagement of this co-stimulatory receptor leads to elevated levels of cytokine secretion as well as the upregulation of the antiapoptotic molecules Bcl-2 and Bcl-xl. This results in increased proliferation and protection against activation-induced T cell death which is also critical for the formation of immunological memory [19, 20]. 4-1BB expression in tumor infiltrating T cells (TIL) [21], coupled with its capacity to promote survival, expansion, and enhanced effector function of activated T cells, has made it an alluring target for cancer immunotherapy. Indeed, stimulation of the co-stimulatory pathway 4-1BB/4-1BBL is beneficial in many therapeutic cancer settings including mono- or combination-therapies with agonistic 4-1BB antibodies or 4-1BBL-expressing viral vectors [22]. 4-1BB co-stimulation is also central for third-generation Chimeric Antigen Receptor (CAR) T cell therapy, as its endodomain is incorporated into the tumor binding chimeric receptor to enhance signaling and consequently tumor cell killing [23].

Nevertheless, despite the notable progress achieved so far, there is still a need in further medical uses of oncolytic virotherapy.

### SUMMARY OF INVENTION

It is an object of the present invention to provide further medical uses of local, i.e. tumor directed virotherapy using oncolytic viruses.

Briefly, the object of the present invention is solved by a recombinant MVA expressing a TAA and the costimulatory molecule 4-1BBL for use in
(i) the prevention of recurrence of a solid tumor, wherein the recombinant MVA is intratumorally administered to the solid tumor, or
(ii) the treatment, prevention and/or prevention of recurrence of a tumor, wherein the recombinant MVA is intratumorally administered to another solid tumor.

In particular, the invention is defined by the appended claims and by the following aspects and their embodiments.

In one aspect, the invention provides a recombinant MVA comprising:
(a) a first nucleic acid encoding a tumor-associated antigen (TAA), and
(b) a second nucleic acid encoding a 4-1BB ligand (4-1BBL),

for use in the treatment and prevention of recurrence of a solid tumor in a subject, wherein the recombinant MVA is locally, preferably intratumorally, administered to the solid tumor.

In a further aspect, the invention provides a recombinant MVA comprising:
(a) a first nucleic acid encoding a tumor-associated antigen (TAA), and
(b) a second nucleic acid encoding a 4-1BB ligand (4-1BBL),

for use in the prevention of recurrence of a solid tumor in or after its remission in a subject, wherein the remission is induced by or follows local, preferably intratumoral, administration of the recombinant MVA to the solid tumor.

In another aspect, the invention provides a recombinant MVA comprising:
(a) a first nucleic acid encoding a tumor-associated antigen (TAA), and
(b) a second nucleic acid encoding a 4-1BB ligand (4-1BBL),

for use in the treatment, prevention and/or prevention of recurrence of a secondary tumor in a subject, wherein the recombinant MVA is locally, preferably intratumorally, administered to a related primary solid tumor, but is not administered to the secondary tumor; or

for use in the treatment and/or prevention of recurrence of a primary solid tumor in a subject, wherein the recombinant MVA is locally, preferably intratumorally, administered to a related secondary solid tumor, but is not administered to the primary solid tumor; or

for use in the treatment, prevention and/or prevention of recurrence of a secondary tumor in a subject, wherein the recombinant MVA is locally, preferably intratumorally, administered to another related secondary solid tumor, but is not administered to the first mentioned secondary tumor.

In yet another aspect, the invention provides a recombinant MVA comprising:
(a) a first nucleic acid encoding a tumor-associated antigen (TAA), and
(b) a second nucleic acid encoding a 4-1BB ligand (4-1BBL),

for use in the treatment, prevention and/or prevention of recurrence of a tumor in a subject, which tumor is in not dissectible or not accessible by surgery, wherein the recombinant MVA is locally, preferably intratumorally, administered to another related solid tumor, but is not locally, preferably intratumorally, administered to the first mentioned tumor.

In yet another aspect, the invention provides a recombinant MVA comprising:
(a) a first nucleic acid encoding a tumor-associated antigen (TAA), and
(b) a second nucleic acid encoding a 4-1BB ligand (4-1BBL),

for use in the induction of a systemic anti-tumor immune response in a subject, wherein the recombinant MVA is locally, preferably intratumorally, administered to a solid tumor.

In yet another aspect, the invention provides a recombinant MVA comprising:
(a) a first nucleic acid encoding a tumor-associated antigen (TAA), and
(b) a second nucleic acid encoding a 4-1BB ligand (4-1BBL),

for use in a systemic anti-tumor therapy, wherein the recombinant MVA is locally, preferably intratumorally, administered to a solid tumor.

In yet another aspect, the invention provides a recombinant MVA comprising:
(a) a first nucleic acid encoding a tumor-associated antigen (TAA), and
(b) a second nucleic acid encoding a 4-1BB ligand (4-1BBL),

for use in a neoadjuvant anti-tumor therapy, wherein the recombinant MVA is locally, preferably intratumorally, administered to a solid tumor.

In yet another aspect, the invention provides a recombinant MVA comprising:
(a) a first nucleic acid encoding a tumor-associated antigen (TAA), and
(b) a second nucleic acid encoding a 4-1BB ligand (4-1BBL),

for use in the induction of an anti-tumor immunological memory in a subject, wherein the recombinant MVA is locally, preferably intratumorally, administered to a solid tumor.

In yet another aspect, the invention provides a recombinant MVA comprising:
(a) a first nucleic acid encoding a tumor-associated antigen (TAA), and
(b) a second nucleic acid encoding a 4-1BB ligand (4-1BBL),

for use in the treatment of a plurality of tumors in a subject, wherein the recombinant MVA is locally, preferably intratumorally, administered to at least one, but not all, of said plurality of tumors.

The invention also provides methods of treatment for a patient having a plurality of tumors comprising locally (preferably intratumorally) administering a recombinant MVA to at least one, but not all, of said plurality of tumors, wherein the recombinant MVA comprises a first nucleic acid encoding a tumor-associated antigen (TAA) and a second nucleic acid encoding a 4-1-BB ligand (4-1-BBL).

These aspects and their embodiments will be described in further detail in connection with the description of invention.

### DESCRIPTION OF DRAWINGS/FIGURES

FIGS. 1A-1F show that therapeutic efficacy of intratumoral administration of MVA-TAA-4-1BBL in unrelated, large tumor models is independent of the choice of antigen.

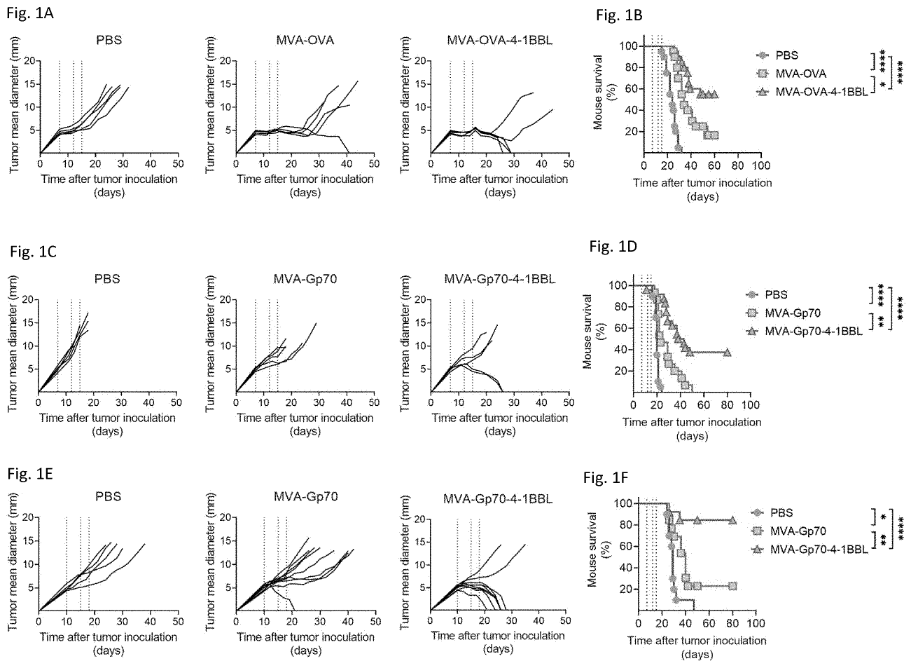

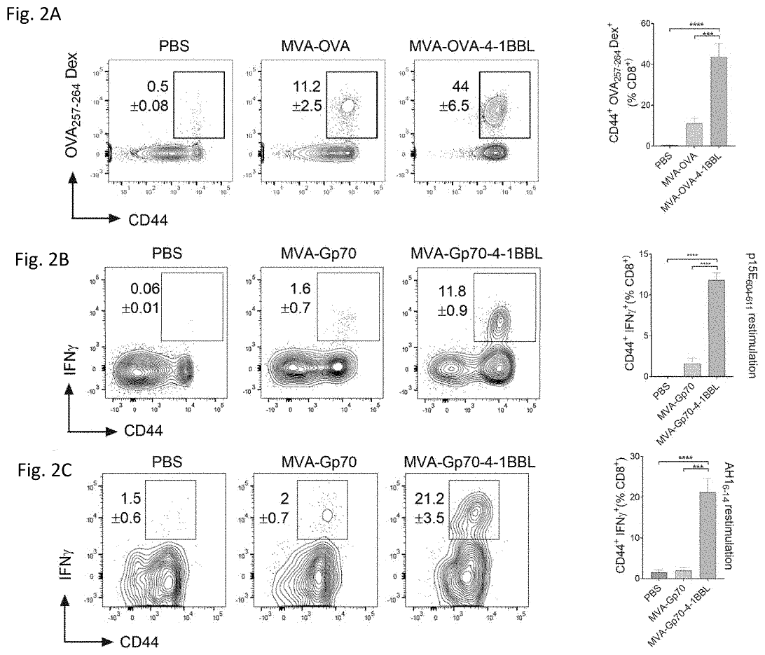

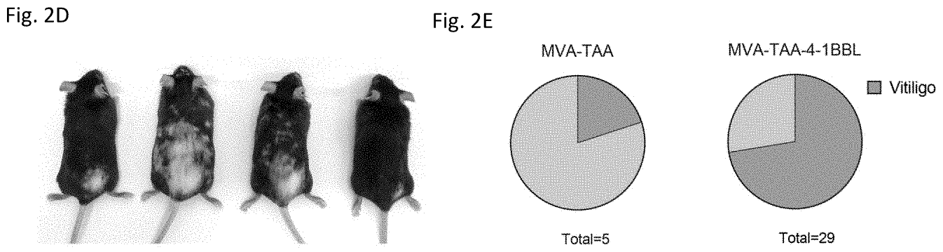

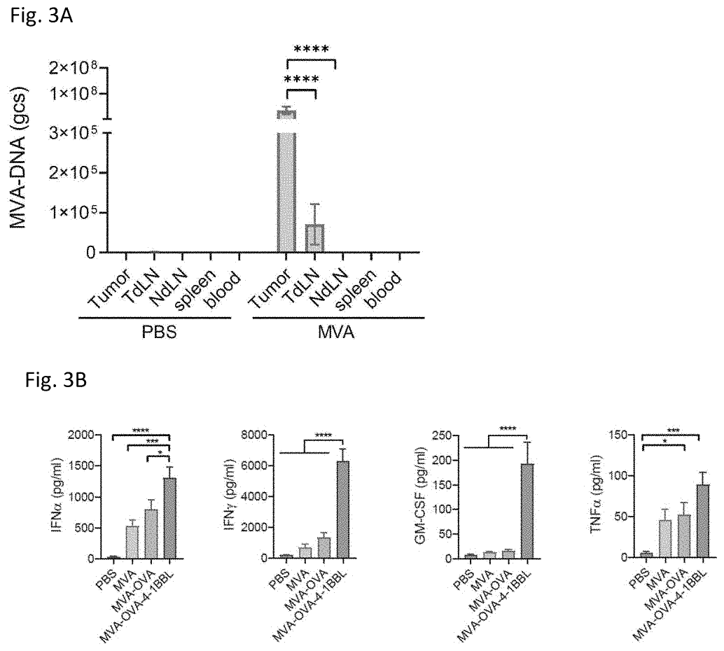

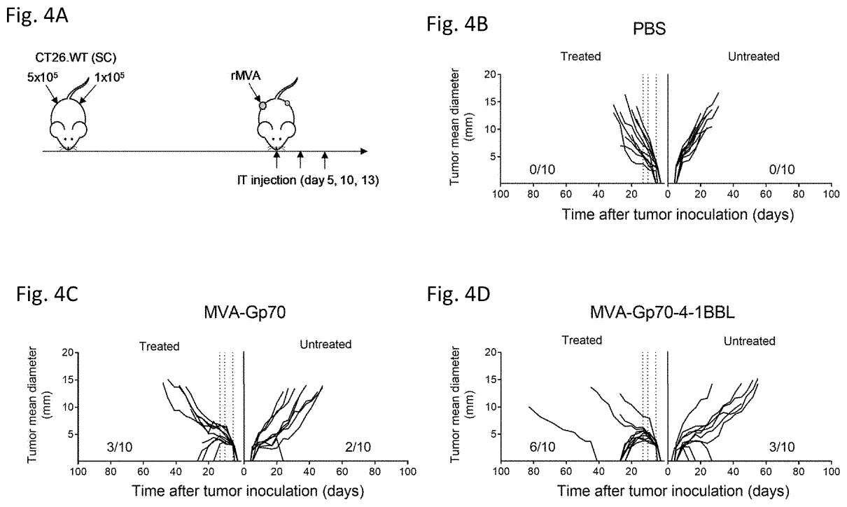

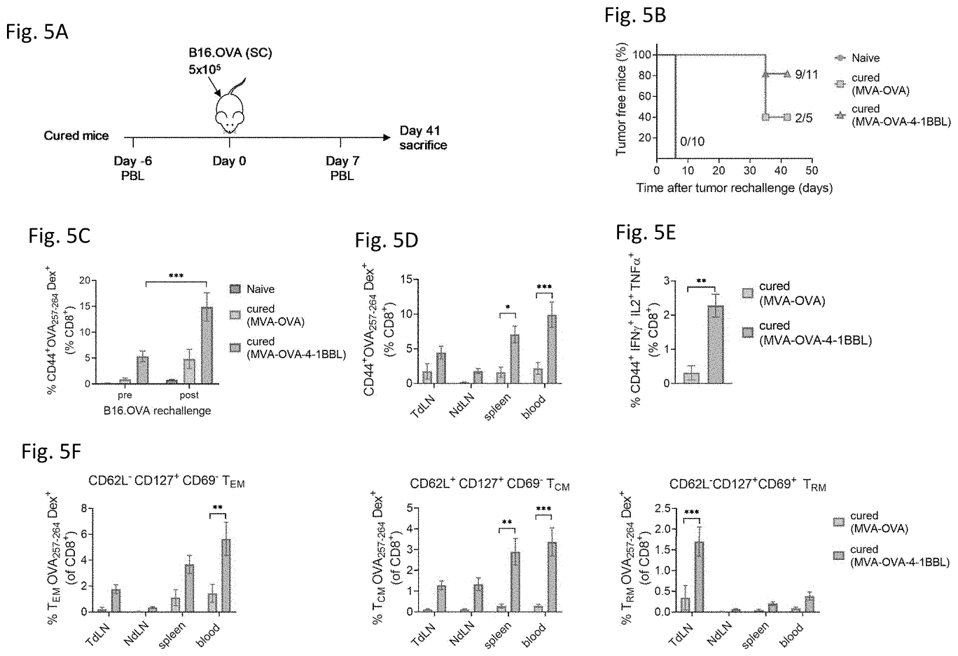

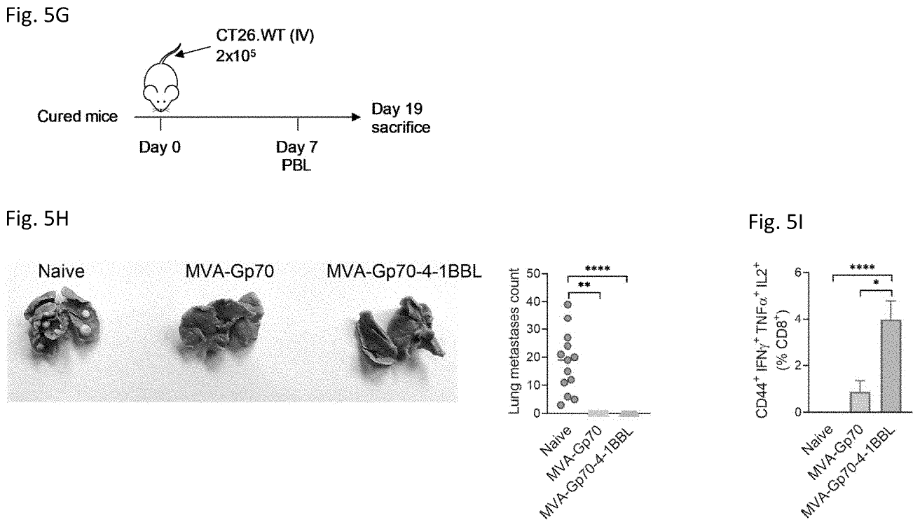

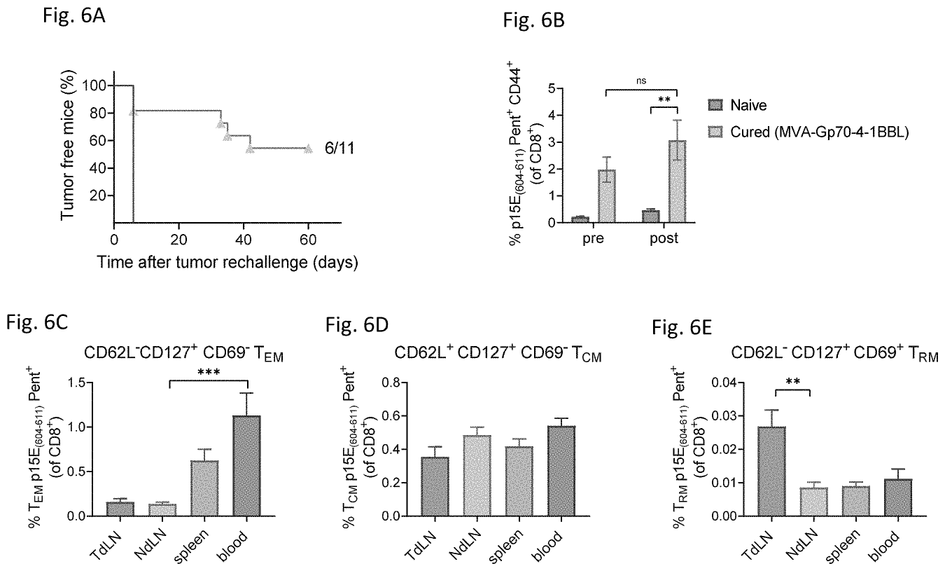

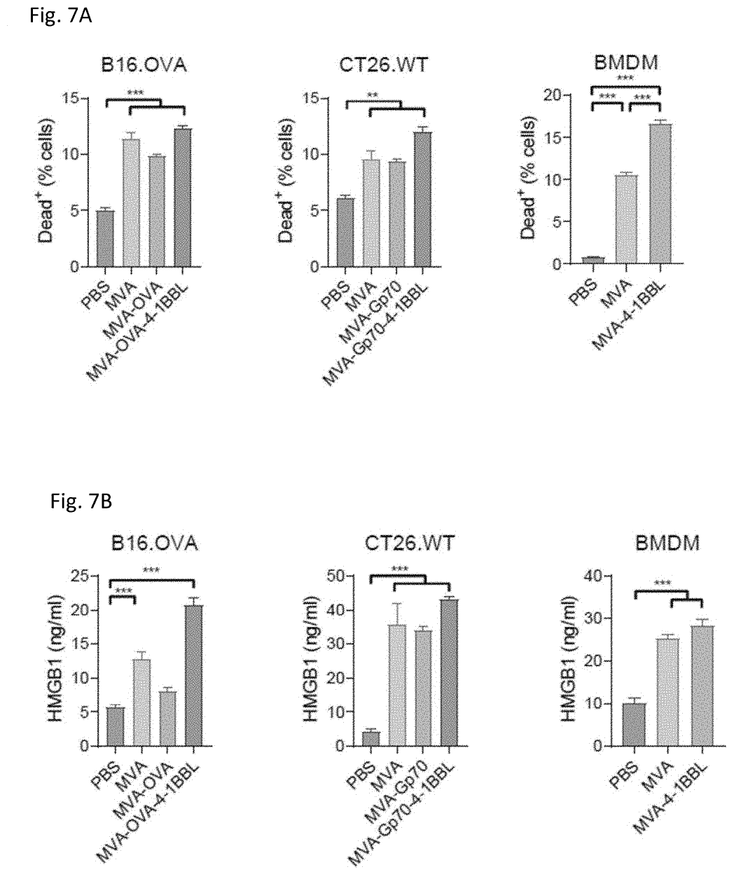

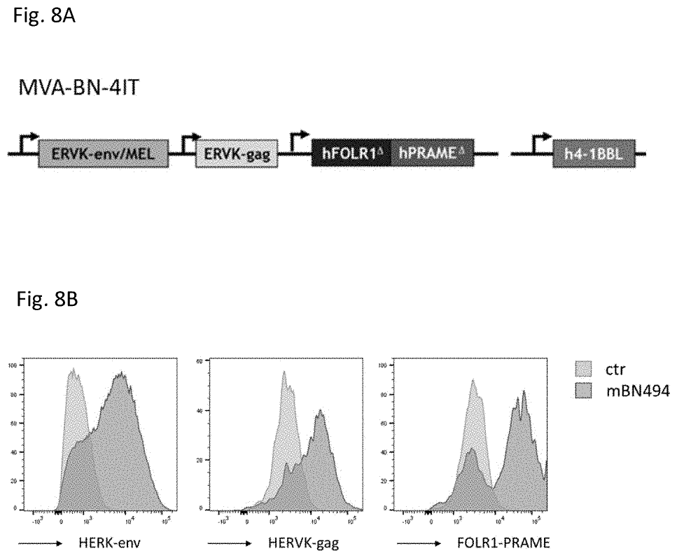

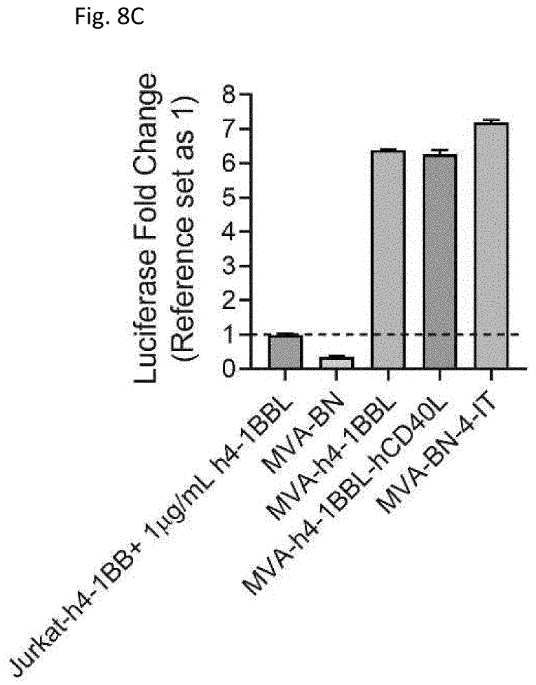

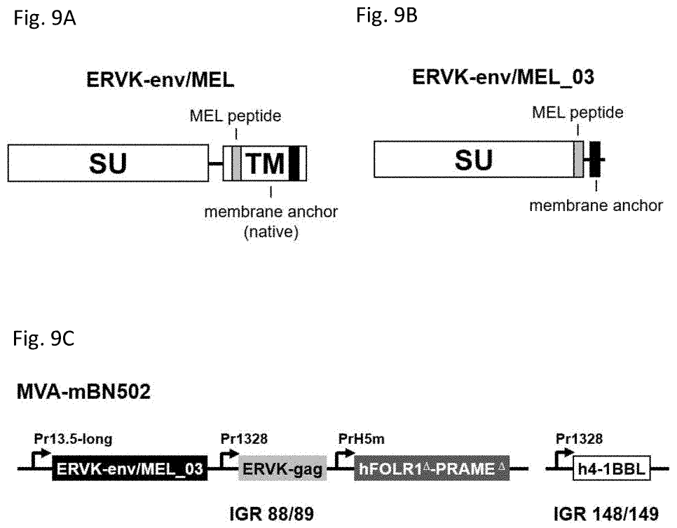

C57BL/6 (FIG. 1A-1D) or Balb/c mice (FIG. 1E-1F) received either 5×10⁵ B16.OVA (FIG. 1A-1B), 5×10⁵ B16.F10 (FIG. 1C-1D) or 5×10⁵ CT26.WT (FIG. 1E-1F) cells subcutaneously (SC) in the flank. Seven to fourteen days later, when tumors were above 60 mm³, mice were immunized intratumorally (IT) either with PBS or with the indicated MVA constructs. IT immunization was repeated on days 4 or 5 and 8 after the first immunization (dashed lines). (FIG. 1A) Tumor size follow-up (n=5 mice/group) and (FIG. 1B) overall survival (n=20 mice/group) of B16.OVA bearing mice injected either with PBS, 2×10⁸ TCID₅₀ MVA-OVA or 2×10⁸ TCID₅₀ MVA-OVA-4-1BBL; (FIG. 1C) Tumor size follow-up (n=5 mice/group) and (FIG. 1D) overall survival (n=15 mice/group) of B16.F10 bearing mice injected either with PBS, 5×10⁷ TCID₅₀ MVA-Gp70 or 5×10⁷ TCID₅₀ MVA-Gp70-4-1BBL; (FIG. 1E) Tumor size follow-up (n=5 mice/group) and (FIG. 1F) overall survival (n=10 mice/group) of CT26.WT bearing mice injected either with PBS, 5×10⁷ TCID₅₀ MVA-Gp70 or 5×10⁷ TCID₅₀ MVA-Gp70-4-1BBL. (FIGS. 1A, 1C and 1E) data are representative of at least two independent experiments. (FIGS. 1B, 1D and 1F) represent overall survival of at least 2 merged independent experiments. Log rank test on mouse survival was performed for figures B, D and F. *, p<0.05; **, p<0.005; ****, p<0.0001.

FIG. 2A-2E show increased peripheral blood CD8⁺ T cell responses in MVA-TAA-4-1BBL immunized tumor-bearing mice.

(FIG. 2A) Representative dot plots and frequency of peripheral blood CD44⁺ OVA₂₅₇₋₂₆₄ Dex⁺ CD8⁺ T cells 3 days after last IT PBS, MVA-OVA or MVA-OVA-4-1BBL immunization of B16.OVA tumor-bearing mice (n=5 mice/group). (FIG. 2B) Representative dot plots and frequency of p15E₆₀₄₋₆₁₁ peptide restimulated peripheral blood CD44⁺ IFNγ⁺ CD8⁺ T cells 3 days after last IT PBS, MVA-Gp70 or MVA-Gp70-4-1BBL immunization of B16.F10 tumor-bearing mice (n=5 mice/group). (FIG. 2C) Representative dot plots and frequency of AH1₆₋₁₄ peptide restimulated peripheral blood CD44⁺ IFNγ⁺ CD8⁺ T cells 3 days after last IT PBS, MVA-Gp70 or MVA-Gp70-4-1 BBL immunization of CT26.WT tumor-bearing mice (n=5 mice/group). (FIG. 2D) Representative picture of vitiligo development in IT MVA-TAA-4-1 BBL cured C57BL/6 mice. (FIG. 2E) Pie chart displaying vitiligo incidence of IT MVA-TAA (data combined from MVA-OVA and MVA-Gp70) and MVA-TAA-4-1BBL (data combined from MVA-OVA-4-1 BBL and MVA-Gp70-4-1BBL combined) cured C57BL/6 mice, respectively. (FIG. 2A-2E) Data are representative of at least two independent experiments. (FIG. 2A-2C) Data expressed as Mean±SEM. One-way ANOVA was performed. ***p<0.005, ****p<0.001.

FIG. 3A-3B show MVA localization upon IT MVA injection and induction of inflammatory cytokines by MVA-OVA and MVA-OVA-4-1-BBL.

(FIG. 3A) C57BL/6 mice received 5×10⁵ B16.OVA cells SC. When tumors reached 60 mm³, mice were grouped (n=3 mice/group) and administered IT either with PBS or with 2×10⁸ TCID₅₀ MVA (MVA not encoding a TAA or 4-1-BBL). 6 hours after IT injection tumor, TdLN, NdLN, spleen and blood were snap frozen and viral DNA was extracted from tissue lysates. Gene Copies (gcs) of the MVA gene MVA082L in the different organs is shown. Data expressed as Mean±SEM. Two-way ANOVA was performed. *, p<0.05; ***, p<0.005; ****, p<0.0001. (FIG. 3B) C57BL/6 mice received 5×10⁵ B16.OVA cells. When tumors reached 60 mm³, mice were grouped (n=3 mice/group) and administered IT either with PBS or with 2×10⁸ TCID₅₀ MVA, MVA-OVA or MVA-OVA-4-1 BBL. 6 hours after IT injection tumors were extracted and tumor lysates processed. Concentration (pg/ml) of indicated cytokines/chemokines in tumor lysates is shown. Data expressed as Mean±SEM. One-way ANOVA was performed. *, p<0.05; **, p<0.01; ****, p<0.0001.

FIG. 4A-4D show that intratumoral MVA-immunotherapy induces rejection of untreated lesions.

(FIG. 4A-4D) Bilateral tumor model. (FIG. 4A) Experimental layout. Balb/c mice received 5×10⁵ and 1×10⁵ CT26.WT tumor cells SC into the right and left flank, respectively. 5 days later right flank tumors were immunized IT either with PBS or with the indicated MVA constructs. IT immunization was repeated on days 5 and 8 after the first immunization (arrows). (FIG. 4B) Tumor size follow-up (n=10 mice/group) of the treated and untreated tumor after PBS IT injection. (FIG. 4C) Tumor size follow-up (n=10 mice/group) of the treated and untreated tumor after 5×10⁷ TCID₅₀ MVA-gp70 IT injection. (FIG. 4D) Tumor size follow-up (n=10 mice/group) of the treated and untreated tumor after 5×10⁷ TCID₅₀ MVA-Gp70-4-1BBL IT injection.

FIG. 5A-5I show that intratumoral MVA-TAA-4-1BBL treated mice are resistant to subsequent local and systemic tumor re-challenge.

(FIG. 5A) Experimental layout. Naïve C57BL/6 mice or long-term survivors (12-36 weeks after tumor clearance) of FIGS. 1A and 1B were rechallenged SC into the tumor-naïve flank of cured mice with 5×10⁵ B16.OVA cells. Peripheral blood was analysed by flow cytometry before (day −6) and after (day 7) after rechallenge. Blood, spleen, non-dLN and TdLN mononuclear cells were analysed on day 41 after tumor cell inoculation. (FIG. 5B) Percentage of tumor-free mice over time is displayed (n=5-11 mice/group). In brackets number of tumor free mice per group is shown. (FIG. 5C) Frequency of peripheral blood CD44⁺ OVA₂₅₇₋₂₆₄ Dex⁺ CD8⁺ pre and post B16.OVA rechallenge of naïve mice and long-term survivors after IT MVA-OVA or MVA-OVA-4-1BBL treatment. (FIG. 5D) Frequency of CD44⁺ OVA₂₅₇₋₂₆₄ Dex⁺ CD8⁺ T in blood, spleen, NdLN and TdLN. (FIG. 5E) Frequency of splenic CD44⁺ IFNγ⁺ TNFα⁺ IL2⁺ CD8⁺ T cells after restimulation with OVA₂₅₇₋₂₆₄ peptide. (FIG. 5F) Frequency of CD62L⁻ CD127⁺ CD69⁻ OVA₂₅₇₋₂₆₄ Dex⁺ cells (T_{EM}) in blood, spleen, NdLN and TdLN (left). Frequency of CD62L⁺ CD127⁺ OVA₂₅₇₋₂₆₄ Dex⁺ cells (T_{CM}) in blood, spleen, NdLN and TdLN (middle). Frequency of CD62L⁻ CD127⁺ CD69⁺ OVA₂₅₇₋₂₆₄ Dex⁺ (T_{RM}) in blood, spleen, NdLN and TdLN 41 days after B16.OVA cell challenge (right). (FIG. 5G-5I) Systemic tumor rechallenge. (FIG. 5G) Experimental layout. Naïve Balb/c mice or long-term survivors of FIG. 1C were rechallenged IV with 2×10⁵ CT26.WT cells. Spleen and lungs were analysed on day 19 after tumor cell injection. (FIG. 5H) Representative photographs of lungs after fixation in Bouin's solution on day 19 after tumor cell transfer into naïve or cured mice. Total number of macroscopic pulmonary metastasis was evaluated (n=2-9 mice/group). (FIG. 5I) Frequency of splenic CD44⁺ IFNγ⁺ TNFα⁺ IL2⁺ CD8⁺ T cells after restimulation with AH1₆₋₁₄ peptide 19 days after rechallenge. (FIG. 5A-5H) n=5-11 mice/group. (FIG. 5C-5H) Data are expressed as Mean±SEM. Two-way ANOVA was performed *, p<0.05; **, p<0.01; ***, p<0.005. (FIG. 5I) Data are expressed as Mean±SEM. One-way ANOVA was performed *, p<0.05; **, p<0.005; ***, p<0.0005.

FIG. 6A-6E show that intratumoral MVA-Gp70-4-1BBL immunotherapy confers protection from local tumor re-challenge.

Naïve C57BL/6 mice or long-term survivors of FIGS. 1C and 1D were rechallenged SC into the tumor-naïve flank of cured mice with 5×10⁵ B16.F10 cells. Peripheral blood was analysed by flow cytometry before (day −6) and after (day 7) after rechallenge. Blood, spleen, NdLN and TdLN were analysed on day 42 after tumor cell inoculation. (FIG. 6A) Percentage of tumor-free mice over time is displayed (n=5-11 mice/group). In brackets number of tumor free mice per group is shown. (FIG. 6B) Frequency of peripheral blood CD44⁺ p15E₆₀₄₋₆₁₁ Pent⁺ CD8⁺ T cells pre and post B16.F10 cell rechallenge. (FIG. 6C) Frequency of CD62L⁺ CD127⁺ p15E₆₀₄₋₆₁₁ Pent⁺ CD8⁺ T cells (T_{CM}) in blood, spleen, NdLN and TdLN. (FIG. 6D) Frequency of CD62L⁻ CD127⁺ p15E₆₀₄₋₆₁₁ Pent⁺ CD8⁺ T cells (T_{EM}) in blood, spleen, NdLN and TdLN. (FIG. 6E) Frequency of CD62L⁻ CD127⁺ CD69⁺ p15E₆₀₄₋₆₁₁ Pent⁺ CD8⁺ T cells (T_{EM}) in blood, spleen, NdLN and TdLN. (FIG. 6A-6E) n=5-11 mice/group. (FIG. 6B-6E) Data are expressed as Mean±SEM. (FIG. 6C-6E) One-way ANOVA was performed **, p<0.005; *** p<0.0005.

FIG. 7A-7B show that replicating viruses induce death of infected tumor cells and immune cells [33-35].

Infection with MVA, MVA-TAA or MVA-TAA-4-1BBL enhanced B16.OVA and CT26.WT tumor cell death in vitro (FIG. 7A). Macrophages which have been shown to be preferentially infected by MVA [36] were effectively killed, whereby this effect was significantly increased by 4-1BBL adjuvant (FIG. 7A). Cell death results in the release of intracellular proteins such as High Molecular Group Box 1 (HMGB1) that are sensed by innate immune cells and contribute to the initiation of immune responses [37, 38]. A significant increase of HMGB1 was detected after MVA infection of tumor cells or macrophages irrespective of 4-1BBL (FIG. 7B).

FIG. 8A-8C illustrate MVA-based vector MVA-HERV-FOLR1-PRAME-h4-1-BBL (“MVA-mBN494” or “MVA-BN-4IT”) (FIG. 8A) and furthermore shows the vector's capability of loading TAA into HLA of infected cells (FIG. 8B) as well as of expressing h4-1-BBL in a functional, i.e. h4-1-BB receptor binding form (FIG. 8C). For more details, see Example 7.

FIG. 9A-9C illustrate MVA-based vector “MVA-mBN502” (FIG. 9C) and furthermore shows schematic maps of ERVK-env/MEL (FIG. 9A; as used in MVA-mBN494) and ERVK-env/MEL_03 (FIG. 9B; as used in MVA-mBN502).

### DESCRIPTION OF INVENTION

In this study we combined the immune-stimulatory properties of TAA-encoding MVA with the exquisite T cell enhancing potential of 4-1BBL and evaluated therapeutic efficacy against solid tumors. We found that IT injection of MVA-TAA-4-1BBL exerted strong objective therapeutic responses in various unrelated tumor models. The therapy was due to strongly re-activated tumor-specific CD8⁺ T cells and the favorable induction of multiple proinflammatory chemokines and cytokines in the TME. Furthermore, IT MVA-TAA-4-1BBL injection induced systemic anti-tumor immune responses inhibiting growth of tumor deposits at distant sites. Importantly, IT MVA-TAA-4-1BBL triggered the generation of a diversified tumor-specific memory response that protected against local and metastatic recurrence.

We cloned tumor associated antigens (TAA) and the immune-stimulatory adjuvant 4-1BBL into the genome of modified vaccinia Ankara (MVA) for intratumoral virotherapy. Local treatment with MVA-TAA-4-1BBL resulted in control of established tumors. Upon intratumoral injection, MVA localized mainly to the tumor with minimal leakage to the tumor draining lymph node. In situ infection by MVA-TAA-4-1BBL triggered profound changes in the tumor microenvironment including the induction of multiple proinflammatory molecules and immunogenic cell death. This led to the reactivation and expansion of antigen-experienced, cytokine producing tumor-specific cytotoxic T cells. Strikingly, we report the induction of a systemic anti-tumor immune response by local MVA-TAA-4-1BBL treatment that controlled tumor growth at distant, untreated lesions and conferred protection against local and systemic tumor rechallenge. In all cases 4-1BBL adjuvanted MVA was superior to MVA. Hence, intratumoral 4-1BBL-adjuvanted MVA immunotherapy induced expansion of potent tumor-specific CD8⁺ T cells as well as favorable proinflammatory changes in the tumor microenvironment leading to elimination of tumors and protective immunological memory.

### Definitions

It must be noted that, as used herein, the singular forms “a”, “an”, and “the”, include plural references unless the context clearly indicates otherwise. Thus, for example, reference to “a nucleic acid sequence” includes one or more nucleic acid sequences.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the appended claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used in the context of an aspect or embodiment in the description of the present invention the term “comprising” can be amended and thus replaced with the term “containing” or “including” or when used herein with the term “having.” Similarly, any of the aforementioned terms (comprising, containing, including, having), whenever used in the context of an aspect or embodiment in the description of the present invention include, by virtue, the terms “consisting of” or “consisting essentially of,” which each denotes specific legal meaning depending on jurisdiction.

When used herein “consisting of” excludes any element, step, or ingredient not specified in the claim element. When used herein, “consisting essentially of” does not exclude materials or steps that do not materially affect the basic and novel characteristics of the claim.

The term “recombinant MVA” refers to MVA having an exogenous nucleic acid sequence inserted in its genome which is not naturally present in the parent virus. A recombinant MVA thus refers to MVA made by an artificial combination of MVA nucleic acid sequences with a segment of nucleic acid sequences of synthetic or semisynthetic origin which combination does not occur or is differently arranged in nature. The artificial combination is most commonly accomplished by artificial manipulation of isolated segments of nucleic acids, using well-established genetic engineering techniques. Generally, a “recombinant MVA” as described herein refers to MVA that is produced by standard genetic engineering methods, e.g., a recombinant MVA is thus a genetically engineered or a genetically modified MVA. The term “recombinant MVA” thus includes MVA (e.g., MVA-BN) which has integrated at least one recombinant nucleic acid, preferably in the form of a transcriptional unit, in its genome. A transcriptional unit may include a promoter, enhancer, terminator and/or silencer. Recombinant MVA of the present invention is designed to express heterologous antigenic determinants, polypeptides or proteins (antigens) upon induction of the regulatory elements e.g., the promoter.

The term “subject”, as used herein, refers to a recipient of the recombinant MVA, who typically is a mammal, such as a non-primate or a primate (e.g. monkey or human), and preferably is a human. The term includes a human or animal “patient” and a laboratory animal. The terms “subject” and “patient” are used interchangeably.

The term “solid tumor” refers to localizable or settled neoplastic tissue, in contrast to, for example, leukemias or hematological cancers.

The term “primary tumor” refers to an initial or first tumor from which neoplastic cells may spread to other parts of the body and may form new (“secondary”) tumors.

The term “secondary tumor”, as used herein, refers to a tumor formed by neoplastic cells spread from a “primary tumor” or from another “secondary tumor”. Particularly, the term includes a “metastasis”.

The term “metastasis” refers to a tumor deposit deriving from a cancerous primary or secondary tumor.

The wording “related”, as used herein, means that two tumors are supposed to be responsive to the same recombinant MVA or TAA. They are, for example, of the same tumor type.

The term “spatially distant”, as used herein, relates to tumors that are clearly discernible as individual tumors, for example by optical means, and spaced from another.

The term “local” administration includes, e.g., intratumoral or topical administration to a solid tumor. Thus, local means on site. The administration can be achieved, for example, by local or topical application or by intratumoral injection of the recombinant MVA. In some embodiments, for example, in a tumor that is close to the skin or is a tumor of skin cells such as melanoma, local administration can be achieved by subcutaneous injection in the immediate vicinity of the tumor. In contrast, terms like “distant” or “systemic” mean not on site.

The term “systemic”, as used herein, means the opposite of “local”. Thus, systemic may mean that an effect is locally unrestricted, i.e. rather relates to the entire body.

The term “tumor directed virotherapy”, as used herein, means a local virotherapy in contrast to systemic tumor virotherapy.

The term “systemic anti-tumor therapy”, as used herein, means that the recombinant MVA is administered locally to a solid tumor, but the anti-tumoral activity extends to other tumors not locally treated.

The term “systemic anti-tumor immune response”, as used herein, refers to an anti-tumor immune response potentially occurring anywhere in the body.

The term “recurrence”, as used herein, refers to a relapse or return of a tumor.

The term “prevention of recurrence”, as used herein, means that a relapse of a remitted tumor, i.e. a tumor in or after remission, for example an eradicated tumor, is prevented or inhibited. At least, the tumor's full recurrence is inhibited, for example at least at 50%.

The term “prevention”, in contrast, means that the first appearance of a tumor is prevented.

The term “remission”, as used herein, relates to a tumor which, for example, is decreased or eradicated. More generally, “remission” is the reduction or disappearance of the signs and symptoms of a disease such as a tumor. The remission may be considered a partial remission or a complete or full remission. For example, a partial remission of a tumor may be defined as a 50% or greater reduction in the measurable parameters of tumor growth as may be found on physical examination, radiologic study, or by biomarker levels from a blood or urine test. A complete remission, on the other hand, is a total disappearance of the tumor, notwithstanding the possibility of a relapse.

The term “neoadjuvant therapy” refers to a treatment prior to the main therapy. Here, a neoadjuvant therapy using the recombinant MVA aims to reduce the size or extent of a tumor before, e.g., surgical excision.

### Selected Abbreviations

IT intratumoral (i.t.)
MVA Modified Vaccinia Virus Ankara (MVA)
OV oncolytic virus
TAA tumor associated antigen
TME tumor microenvironment

### EMBODIMENTS

In one embodiment, the recombinant MVA is a non-replicating or replication deficient MVA. Preferably, the recombinant MVA is not capable of reproductive replication in human cell lines.

In one embodiment, the recombinant MVA is derived from MVA-BN as deposited at the European Collection of Cell Cultures (ECACC) under number V00083008, or from an MVA-BN derivative.

In one embodiment, the recombinant MVA comprises:
(a) a first nucleic acid encoding a tumor-associated antigen (TAA); and
(b) a second nucleic acid encoding a 4-1BB ligand (4-1BBL); and
(c) at least one further nucleic acid encoding a TAA.

In one embodiment, the first nucleic acid encoding a TAA and the at least one further nucleic acid encoding a TAA are the same or different.

In one embodiment the recombinant MVA comprises two, three, four, five, six, or more nucleic acids each encoding a different TAA.

In one embodiment, the TAA is a neoantigen or an endogenous self-antigen.

In one embodiment, the TAA is a transposable element (TE).

In one embodiment, the TAA is an endogenous retroviral protein (HERV).

In one embodiment, the TAA is a long interspersed nuclear element (LINE).

In one embodiment, the TAA is a short interspersed nuclear element (SINE).

In one embodiment, the TAA is selected from the group consisting of an endogenous retroviral (ERV) protein, an endogenous retroviral (ERV) peptide, carcinoembryonic antigen (CEA), mucin 1 cell surface associated (MUC-1), prostatic acid phosphatase (PAP), prostate specific antigen (PSA), human epidermal growth factor receptor 2 (HER-2), survivin, tyrosine related protein 1 (TRP1), tyrosine related protein 1 (TRP2), Brachyury, folate receptor alpha (FOLR1), preferentially expressed antigen of melanoma (PRAME), and the endogenous retroviral peptide MEL; and combinations thereof. Any TAA is suitable for use in the compositions and/or methods of the invention so long as it is an antigen associated with a known tumor or a tumor in the patient to be treated and is capable, when expressed, of giving rise to or stimulating an immune response.

In one embodiment, the ERV protein is from the human endogenous retroviral K (HERV-K) family, preferably is selected from a HERV-K envelope (HERV-K-env) protein and a HERV-K gag protein.

In one embodiment, the ERV peptide is from the human endogenous retroviral K (HERV-K) family, preferably is selected from a pseudogene of a HERV-K envelope protein (HERV-K-env/MEL).

In one embodiment, the recombinant MVA comprises:
(i) a nucleic acid encoding HERV-K-env/MEL;
(ii) a nucleic acid encoding HERV-K gag;
(iii) a nucleic acid encoding FOLR1 and PRAME, preferably expressed as a fusion protein; and
(iv) a nucleic acid encoding 4-1BBL.

In one embodiment, the nucleic acid in (i) encodes a HERV-K-env/MEL comprising a HERV-K env surface (SU) unit and a HERV-K transmembrane (TM) unit, wherein the HERV-K TM unit is mutated, preferably wherein the HERV-K TM unit is mutated such that an immunosuppressive domain is inactivated. Preferably, HERV-K-MEL is inserted within the mutated HERV-K TM unit. More preferably, HERV-K-MEL replaces a portion of the immunosuppressive domain of HERV-K TM.

In one embodiment, the nucleic acid sequence in (i) encodes an amino acid sequence comprising or consisting of an amino acid sequence as depicted in SEQ ID NO: 7.

In one embodiment, the nucleic acid sequence in (i) comprises or consists of a nucleic acid sequence as depicted in SEQ ID NO: 8.

In one embodiment, the nucleic acid in (i) encodes a HERV-K-env/MEL comprising a HERV-K env surface (SU) unit and a HERV-K transmembrane (TM) unit, wherein the HERV-K TM unit is shortened to less than 20 amino acids, preferably less than 10 amino acids, more preferably less than 8 amino acids, most preferably 6 amino acids.

In one embodiment, the nucleic acid in (i) encodes a HERVK-env/MEL comprising a HERV-K-env surface (SU) unit, wherein the RSKR furin cleavage site of the HERV-K-env SU unit is deleted. Preferably, HERV-K-MEL is attached to the C-terminus of the HERV-K-env SU unit.

In one embodiment, the nucleic acid in (i) encodes a HERVK-env/MEL comprising a heterologous membrane anchor, preferably derived from the human PDGF (platelet-derived growth factor) receptor.

In one embodiment, the nucleic acid sequence in (i) encodes an amino acid sequence comprising or consisting of an amino acid sequence as depicted in SEQ ID NO: 11.

In one embodiment, the nucleic acid sequence in (i) comprises or consists of a nucleic acid sequence as depicted in SEQ ID NO: 12.

In one embodiment, the solid tumor, primary solid tumor and/or secondary tumor is a cancerous or malignant tumor.

In one embodiment, the solid tumor, primary solid tumor and/or secondary tumor is a melanoma or a malignant breast, colon or ovarian tumor.

In one embodiment, the secondary tumor is a metastasis.

In one embodiment, the secondary tumor is a solid tumor. Alternatively, the secondary tumor is, for example, an aggregation of tumor cells floating in a bodily fluid such as blood lymph, or is present within a body cavity, e.g. the peritoneal cavity. A primary or secondary tumor to which the recombinant MVA is locally or intratumorally administered, however, is always a solid tumor.

In one embodiment, the tumor to which the recombinant MVA is not locally or intratumorally administered is not dissectible or not accessible by surgery.

In one embodiment, the primary and secondary tumors are spatially distant from each other.

In one embodiment, the secondary tumor and another secondary tumor are spatially distant from each other.

In one embodiment, the primary and secondary tumors are located within the same tissue or organ of a subject's body.

In one embodiment, the secondary tumor and another secondary tumor are located within the same tissue or organ of a subject.

In one embodiment, the primary and secondary tumors are located within different tissues or organs of a subject's body.

In one embodiment, the secondary tumor and another secondary tumor are located within different tissues or organs of a subject's body. In one embodiment, the secondary tumor is naïve, i.e. was not locally, preferably intratumorally, treated with the recombinant MVA before.

In one embodiment, the tumor is in at least partial remission, e.g. in at least more than 50% remission, preferably is in complete remission.

In one embodiment, the recombinant MVA is not administered other than locally, preferably intratumorally. Thus, the recombinant MVA is not administered, e.g., systemically, intravenously, peritoneally, parentally, subcutaneously, or intranasally to the subject.

In one embodiment, the anti-tumor immunological memory is long-term, i.e. lasting for days, weeks, months, years or decades.

### Methods of Treatment

The invention provides methods of treatment for a subject having more than one tumor in which fewer than all of the tumors in the subject are treated by local administration of the recombinant MVA. That is, the invention provides methods of treatment for a subject having a plurality of tumors comprising locally administering a recombinant MVA to at least one, but not all, of said plurality of tumors. In some embodiments, the step of locally administering a recombinant MVA comprises intratumoral injection. In this manner, the methods of treatment can be said to produce “treated tumors” or “injected tumors” to which the recombinant MVA has been locally or intratumorally administered or injected and “untreated tumors” or “uninjected tumors” to which the recombinant MVA was not locally administered or into which the recombinant MVA was not injected. The methods of treatment can also be said to provide a method of treating uninjected tumors in a subject by locally administering or intratumorally injecting at least one other tumor in said subject with the recombinant MVA. In some embodiments of the methods of the invention, the subject is a human patient.

In some embodiments, the methods of treatment comprise treating metastases or recurrences of a first or primary tumor by local administration (in some embodiments, by intratumoral injection) of recombinant MVA into said first or primary tumor. In some embodiments, it is not possible to determine which of a plurality of tumors in a subject was the first to occur (i.e., the “primary tumor”), and the method comprises treating at least one of the plurality of tumors by local administration or intratumoral injection of the MVA. Thus, in some embodiments, by “metastasis” or “secondary tumor” is intended that a tumor is in a different location, or has different borders, than a tumor designated as a “first tumor” or “primary tumor.”

The methods of the invention comprise treating at least one of a plurality of tumors in a subject by local administration or intratumoral injection, or treating at least two tumors, at least three, at least four, or more of said tumors, or treating all but one of said plurality of tumors in a subject. In some embodiments, methods of the invention provide that any number of tumors in the subject may be injected or treated by local administration or intratumoral injection with recombinant MVA so long as fewer than all of the tumors in the subject are so injected or treated.

While the invention is not bound by any particular mechanism or mode of action, any method is suitable in the practice of the invention so long as it stimulates an immune response against at least one TAA or tumor in said subject, or cell thereof, or produces a decrease in the volume or size of at least one secondary or uninjected tumor. By “stimulates an immune response” is intended that indicia of a new or increased immune response can be identified in the subject, such as, for example, an increase in the CD8+ T cell population and/or an increase in the amounts of IFN-gamma, TNF-alpha and/or IL-2 produced by the CD8+ T cells in a subject as illustrated in the working examples herein. In this manner, the invention also provides methods of stimulating an immune response to a TAA comprising the step of intratumorally administering a recombinant MVA of the invention to a subject.

In some embodiments, the recombinant MVA comprises: (a) a first nucleic acid encoding a tumor-associated antigen (TAA); and (b) a second nucleic acid encoding a 4-1BB ligand (4-1BBL). The recombinant MVA may further comprise additional nucleic acids encoding additional TAAs. Methods of the invention are suitable for use with a recombinant MVA encoding both at least one TAA and 4-1-BBL. In some embodiments, the recombinant MVA expresses at least one TAA and 4-1-BBL, which can be demonstrated using techniques known in the art.

Sequences of various TAAs and 4-1-BBL that are useful in the methods and compositions of the invention are known in the art and described, for example, in PCT/EP2019/081942 (published as WO 2020/104531) and European Patent Application No. 19210369.5, all of which are incorporated herein by reference in their entirety. An exemplary 4-1-BBL sequence is set forth in NCBI RefSeq NP_003802.1 (and in the amino acid sequence set forth in SEQ ID NO:3, and/or encoded by the nucleic acid sequence set forth in SEQ ID NO:4). Recombinant MVAs useful in the uses and methods of the invention are also described, for example, in European Patent Application No. 19210369.5, incorporated herein by reference.

In certain embodiments, the nucleic acid sequence encoding 4-1-BBL encodes a 4-1-BBL having at least 95%, 96%, 97%, 98%, 99%, or 100% amino acid sequence identity to SEQ ID NO:3. In certain embodiments, the nucleic acid sequence is the sequence set forth in SEQ ID NO:4, or encodes a 4-1-BBL having the amino acid sequence set forth in SEQ ID NO:3.

In certain embodiments, the nucleic acid sequence encoding a TAA encodes a TAA having at least 95%, 96%, 97%, 98%, 99%, or 100% amino acid sequence identity to a previously known TAA, such as, for example, a sequence set forth in the sequence listing provided herewith. In certain embodiments, the nucleic acid sequence encodes a TAA having the amino acid sequence set forth in the sequence listing, or a TAA having an amino acid sequence known in the art.

In some embodiments, at least one individual tumor is measured before and after the local administration (e.g., intratumoral injection) of the recombinant MVA to determine whether the tumor continues to grow, or increases or decreases in size, following the administration. In some methods, additional measurements may be taken at intervals following the administration to track the response of at least one individual tumor. Such measurements may be by any suitable technique and can include visual assessment or external measurement as well as X-ray, ultrasound imaging, intravital microscopy, or any other suitable method known in the art. Measurements of tumors used to assess the response of a tumor to treatment and/or the effectiveness of a treatment can, include, for example, at least one diameter of the tumor and/or an estimation of the volume of the tumor.

In some embodiments, a method of treating a subject having one or more tumors comprises the steps of: (a) obtaining a first measurement of a tumor in said subject; (b) administering a recombinant MVA expressing at least one TAA to one or more, but less than all, of the tumors in said subject to produce treated or injected tumors and untreated or non-injected tumors; (c) obtaining a second measurement of at least one of said untreated or non-injected tumors; and (d) comparing said second measurement to a first measurement of said untreated or non-injected tumor, whereby a decrease in said measurement or a lack of increase in said measurement indicates that the tumor has regressed or decreased in volume or that tumor growth has been delayed. In some embodiments, the recombinant MVA used in these methods expresses at least one TAA and 4-1-BBL, as further discussed elsewhere herein.

In some embodiments, a method of treating a subject having a primary tumor and one or more metastases, or the possibility of metastases (i.e., having a malignant tumor), is intended to prevent new or additional metastases and comprises the steps of: (a) identifying in a subject one or more tumors at risk for metastasis; (b) locally administering a recombinant MVA expressing a TAA to at least one but not all of the tumors in said subject (in some embodiments, by intratumoral injection); (c) detecting the tumors in said subject to confirm that no new tumors or metastases are detectable in said subject. In some embodiments, the recombinant MVA used in these methods expresses a TAA and 4-1-BBL, as further discussed elsewhere herein. In some embodiments, the recombinant MVA used in these methods comprises a heterologous nucleic acid encoding a TAA and a heterologous nucleic acid encoding 4-1-BBL, but does not comprise any additional genes encoded by a heterologous nucleic acid that can affect the immune response of the subject, or that are expected to affect the immune response of a subject, when used to treat a subject as a component of an MVA or separately.

In some embodiments, the invention provides a method of treating an inaccessible tumor in a subject with a plurality of tumors, comprising the step of locally administering recombinant MVA into at least one accessible tumor of the plurality of tumors, wherein the recombinant MVA comprises a first nucleic acid encoding a tumor-associated antigen (TAA) and a second nucleic acid encoding a 4-1-BB ligand (4-1-BBL). In some embodiments, the recombinant MVA comprises at least one additional nucleic acid encoding at least one additional TAA, or at least two, three, or four or more additional nucleic acids encoding at least two, three, or four or more additional TAAs. In some embodiments, the step of locally administering the recombinant MVA comprises intratumoral injection.

By “inaccessible tumor” is intended a tumor that is difficult to treat directly by local administration of recombinant MVA such as by intratumoral injection and/or using other techniques. An “inaccessible tumor” or unresectable tumor may be located in a sensitive area of the body, for example, close to or surrounding important nerves or blood vessels, or in the brain, or in another location where surgery and/or local administration of recombinant MVA may pose a risk of damage to the subject and/or would be difficult to administer. The invention provides methods of treating an inaccessible or unresectable tumor in a subject with a plurality of tumors comprising a step of local administration to a different tumor that is more accessible and/or where local administration is less likely to cause damage to the subject. In some embodiments, the invention provides a method of treating an inaccessible tumor in a subject, comprising: (a) identifying at least one inaccessible tumor and at least one accessible tumor in a subject; and (b) locally administering a recombinant MVA expressing a TAA to at least one accessible tumor in said subject. Optionally, this method further comprises a step of monitoring said inaccessible tumor to confirm that growth of the tumor has slowed or stopped, and/or determining whether said subject has an increased immune response or new immune response subsequent to the administration of the recombinant MVA.

In some embodiments, the invention provides a method of treating a subject having a plurality of malignant tumors comprising locally administering a recombinant MVA expressing a TAA to at least one of said tumors to produce a treated tumor, wherein treatment results in reduction of tumor volume of the treated tumor and at least one other tumor that was not directly treated or injected with the recombinant MVA (also referred to as an “untreated tumor” or “uninjected tumor”).

In some embodiments, by “intratumoral injection” is intended that the administration is made into the tumor, or within the boundaries of the tumor. In some embodiments, by “local administration” is intended that the administration is made in close proximity to the tumor. One of skill in the art is aware that such injections may coincidentally also be characterized as another type of administration such as, for example, parenteral or subcutaneous, depending on the location of the tumor.

### Further Description

### Modified Vaccinia Virus Ankara (MVA)

In the past, MVA was generated by 516 serial passages on chicken embryo fibroblasts of the Ankara strain of vaccinia virus (CVA) (for review see Mayr et al. 1975). This virus was renamed from CVA to MVA at passage 570 to account for its substantially altered properties. MVA was subjected to further passages up to a passage number of over 570. As a consequence of these long-term passages, the genome of the resulting MVA virus had about 31 kilobases of its genomic sequence deleted and, therefore, was described as highly host cell restricted for replication to avian cells (Meyer et al. 1991). It was shown in a variety of animal models that the resulting MVA was significantly avirulent compared to the fully replication competent starting material (Mayr and Danner 1978).

An MVA useful in the practice of the present invention includes MVA-572 (deposited as ECACC V94012707 on 27 Jan. 1994); MVA-575 (deposited as ECACC V00120707 on 7 Dec. 2000), MVA-1721 (referenced in Suter et al. 2009), NIH clone 1 (deposited as ATCC® PTA-5095 on 27 Mar. 2003) and MVA-BN (deposited at the European Collection of Cell Cultures (ECACC) under number V00083008 on 30 Aug. 2000).

More preferably the MVA used in accordance with the present invention includes MVA-BN and MVA-BN derivatives. MVA-BN has been described in WO 02/042480. “MVA-BN derivatives” refer to any virus exhibiting essentially the same replication characteristics as MVA-BN, as described herein, but exhibiting differences in one or more parts of their genomes.

MVA-BN, as well as MVA-BN derivatives, is replication incompetent, meaning a failure to reproductively replicate in vivo and in vitro. More specifically in vitro, MVA-BN or MVA-BN derivatives have been described as being capable of reproductive replication in chicken embryo fibroblasts (CEF), but not capable of reproductive replication in the human keratinocyte cell line HaCat (Boukamp et al 1988), the human bone osteosarcoma cell line 143B (ECACC Deposit No. 91112502), the human embryo kidney cell line 293 (ECACC Deposit No. 85120602), and the human cervix adenocarcinoma cell line HeLa (ATCC Deposit No. CCL-2). Additionally, MVA-BN or MVA-BN derivatives have a virus amplification ratio at least two-fold less, more preferably three-fold less than MVA-575 in Hela cells and HaCaT cell lines. Tests and assay for these properties of MVA-BN and MVA-BN derivatives are described in WO 02/42480 and WO 03/048184.

The term “not capable of reproductive replication” in human cell lines in vitro as described above is, for example, described in WO 02/42480, which also teaches how to obtain MVA having the desired properties as mentioned above. The term applies to a virus that has a virus amplification ratio in vitro at 4 days after infection of less than 1 using the assays described in WO 02/42480 or U.S. Pat. No. 6,761,893.

### Exemplary Generation of a Recombinant MVA Virus

For the generation of a recombinant MVA as disclosed herein, different methods may be applicable. The DNA sequence to be inserted into the virus can be placed into an E. coli plasmid construct into which DNA homologous to a section of DNA of the poxvirus has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of poxvirus DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA viral DNA in the plasmid and the viral genome, respectively, can generate an MVA modified by the presence of foreign DNA sequences, i.e. nucleotides sequences encoding SARS-CoV-2 antigens.

According to a preferred embodiment, a cell of a suitable cell culture as, e.g., CEF cells, can be infected with a MVA virus. The infected cell can be, subsequently, transfected with a first plasmid vector comprising a foreign or heterologous gene or genes, such as one or more of the nucleic acids provided herein, preferably under the transcriptional control of a poxvirus expression control element. As explained above, the plasmid vector also comprises sequences capable of directing the insertion of the exogenous sequence into a selected part of the MVA viral genome. Optionally, the plasmid vector also contains a cassette comprising a marker and/or selection gene operably linked to a poxvirus promoter. The use of selection or marker cassettes simplifies the identification and isolation of the generated recombinant MVA. However, a recombinant poxvirus can also be identified by PCR technology. Subsequently, a further cell can be infected with the recombinant MVA obtained as described above and transfected with a second vector comprising a second foreign or heterologous gene or genes. In case, this gene shall be introduced into a different insertion site of the poxvirus genome, the second vector also differs in the poxvirus-homologous sequences directing the integration of the second foreign gene or genes into the genome of the poxvirus. After homologous recombination has occurred, the recombinant virus comprising two or more foreign or heterologous genes can be isolated. For introducing additional foreign genes into the recombinant virus, the steps of infection and transfection can be repeated by using the recombinant virus isolated in previous steps for infection and by using a further vector comprising a further foreign gene or genes for transfection. There are ample of other techniques known to generate recombinant MVA.

The practice of the invention will employ, if not otherwise specified, conventional techniques of immunology, molecular biology, microbiology, cell biology, and recombinant technology, which are all within the skill of the art. See e.g. Sambrook, Fritsch and Maniatis, Molecular Cloning: A Laboratory Manual, 2nd edition, 1989; Current Protocols in Molecular Biology, Ausubel F M, et al., eds, 1987; the series Methods in Enzymology (Academic Press, Inc.); PCR2: A Practical Approach, MacPherson M J, Hams B D, Taylor G R, eds, 1995; Antibodies: A Laboratory Manual, Harlow and Lane, eds, 1988.

### EXAMPLES

The following examples serve to further illustrate the disclosure. They should not be understood as limiting the invention the scope of which is determined by the appended claims.

### Example 1: Materials and Methods

(i) Generation of MVA Recombinants

The generation of MVA recombinants was carried out as described previously [1]. MVA-BN® was developed by Bavarian Nordic and is deposited at the European Collection of Cell Cultures (ECACC) (V00083008). The generation of a recombinant MVA expressing ovalbumin (MVA-OVA) was previously described [1, 2]. The gene encoding for 4-1BBL was synthetized (Geneart, Life Technologies) and cloned into the MVA-OVA genome to generate MVA-OVA-4-1BBL. The gene encoding the mouse leukemia virus derived envelope glycoprotein Gp70 was synthesized (Geneart, Life Technologies) and cloned into the MVA and MVA-4-1BBL genome, respectively, to generate MVA-Gp70 and MVA-Gp70-4-1BBL. All viruses used in animal experiments were purified twice through a sucrose cushion.

(ii) Ethics Statement

Animal experiments were approved by the animal ethics committee of the government of Upper Bavaria (Regierung von Oberbayern, Sachgebiet 54, Tierschutz) and were carried out in accordance with the approved guidelines for animal experiments at Bavarian Nordic GmbH. The bilateral CT26.WT tumor experiment was conducted at CIMA, University of Navarra (Pamplona, Spain) in compliance with the Association for Assessment and Accreditation of Laboratory Animal Care International (AAALAC).

(iii) Mice and Tumor Cell Lines

6- to 8-week-old female C57BL/6J (H-2^{b}) and Balb/cJ (H-2^{d}) mice were purchased from Janvier Labs. All mice were handled, fed, bred and maintained either in the animal facilities at Bavarian Nordic GmbH, at the University of Zurich or at the University of Navarra according to institutional guidelines.

The B16.OVA melanoma cell line was a kind gift of Roman Spörri (University of Zurich). B16.F10 (ATCC® CRL-6475™) and CT26.WT (ATCC® CRL-2638™) cell lines were purchased from American Type Culture Collection (ATCC). Tumor cells were cultured in DMEM Glutamax medium supplemented with 10% FCS, 1% NEAA, 1% sodium pyruvate and 1% penicillin/streptomycin (all reagents from Gibco) in an incubator at 37° C. 5% CO₂. All tumor cell lines used in experiments conducted at Bavarian Nordic were regularly tested negative for Mycoplasma by PCR.

(iv) Tumor Cell Injection

Mice were injected subcutaneously in the flank with 5×10⁵ tumor cells. Regarding B16.OVA and B16.F10, prior to injection cells were admixed with 7 mg/ml Matrigel (Trevigen). For subcutaneous bilateral tumor experiments, 5×10⁵ and 1×10⁵ CT26.WT tumor cells were injected in the right flank and the left flank respectively. Tumor re-challenge experiments were performed between 3 and 6 months upon clearance of tumors. Subcutaneous re-challenge was carried out at the opposite flank using 5×10⁵ tumor cells. Intravenous re-challenge was performed injecting 2×10⁵ CT26.WT cells. Tumor diameter was measured at regular intervals using a caliper twice a week.

(v) Immunizations

Intratumoral injections were given into the solid tumor mass with a total volume of 50 μl containing the respective MVA recombinants. Repetitive intratumoral injections were performed at days 0, 5 and 8 after tumor grouping, and indicated in the graphs by vertical dotted lines. When indicated, blood was collected 3 days after last intratumoral immunization for peripheral blood immune cell phenotyping.

(vi) Cell Isolation

When indicated, spleens and lymph nodes were harvested from mice. Spleen and lymph node single-cell suspensions were prepared by mechanically disrupting tissues through a 40-μm cell strainer (Falcon). Spleen samples were subjected to red blood cell lysis (Sigma-Aldrich).

Blood was collected in PBS containing 2% FCS, 0.1% sodium azide and 2.5 U/ml heparin. Peripheral blood mononuclear cells (PBMCs) were prepared by lysing erythrocytes with red blood cell lysis buffer. Mononuclear cells from the abovementioned organs were washed, resuspended in RPMI+2% FCS, counted and kept on ice until further analysis. 100 μl TrueCount counting beads (BD Biosciences) were added to the tumor cell suspensions.

(vii) Peptide Restimulation

When indicated, mononuclear cells were incubated with 2.5 pg/ml of MHC class I restricted peptides [OVA₂₅₇₋₂₆₄ (SIINFEKL); p15E₆₀₄₋₆₁₁ (KSPWFTTL); AH1₆₋₁₄ (SPSYVYHQF)] for 5-6 h at 37° C. 5% CO₂ in T cell medium and 10 μg/ml BFA. Peptides were purchased from GenScript.

(viii) Flow Cytometry

Mononuclear cell suspensions, BMDMs or tumor cells were stained for 30 minutes at 4° C. in the dark using fixable live/dead viability kits prior to staining (Life Technologies). Mononuclear cells were stained using antibodies from BD Biosciences, eBioscience or Biolegend. When indicated, cell suspensions were stained using a H-2 k^{b} OVA₂₅₇₋₂₆₄-dextramer (Immudex), a H-2K^{b} p15E₆₀₄₋₆₁₁-pentamer (ProImmune) or a H-2L^{d} AH1₆₋₁₄ pentamer (Prolmmune). For FoxP3 transcription factor and Ki67 staining cells were fixed using FoxP3 Staining Kit (eBioscience). For intracellular cytokine staining, cell suspensions were stained and fixed for intracellular cytokine detection using IC Fixation & Permeabilization Staining kit (eBioscience). All cells were acquired using a digital flow cytometer (LSR II, BD Biosciences) and data were analyzed with FlowJo software version 10.3 (Tree Star).

(ix) Quantitative Real-Time PCR for Quantification of MVA-Specific DNA Genome Copies

Genomic DNA (gDNA) was isolated from tissues using QIAamp DNA Mini Kit according to manufacturer's instructions (Qiagen) and quantified in a NanoVue spectrophotometer (Biochrom). Briefly, a standard curve starting at 5×10⁷ genome copies (gcs) was prepared using a plasmid expressing the open reading frame 082L of MVA, target for detection of MVA backbone DNA. Then, quantitative real-time PCR was performed with TaqMan Gene Expression Master Mix (Thermo-Fisher) using specific primers MVA082L sense 5′-acgtttagccgcctttaatagag-3′, MVA082L antisense 5′-tggtcagaactatcgtcgttgg-3′, and a fluorescein probe 6FAM-aatcccaccgccttctggatctc-BBQ. Calculations were performed by the 7500 software of the Real-time PCR system (Applied Biosystems). The software determines a threshold cycle (C_{T}) for every standard dilution, control and replicate, which is inversely proportional to the logarithm of the quantity of gcs of specific DNA. Based on the standard curve, the software determined the respective number of gcs of the target gene by using the C_{T} value that is measured for each replicate. The quantity (gcs) of a sample is calculated by the average quantity of its duplicate determination.

(x) Statistical Analysis

Statistical analyses were performed as described in the figure legends using GraphPad Prism version 7.02 for Windows (GraphPad Software, La Jolla, Calif.). For immunological data, results are presented as ‘mean’ and ‘standard error of the mean’ (SEM). Either analysis of variance (ANOVA) with multiple comparisons test or one-tailed unpaired Student's t tests were used to determine statistical significance between treatment groups. For tumor-bearing mice survival after treatment, Log rank tests were performed to determine statistical significance between treatment groups.

### Example 2: 4-1BBL Potentiates Intratumoral MVA Immunotherapy

IT application of poxviruses has been shown to effectively induce anti-tumor responses in various tumor models [24-28]. However, most of these studies have been conducted with replicating viruses. Here, we tested whether local treatment of established tumors using the non-replicating poxvirus MVA encoding a Tumor Associated Antigen (TAA defined above) and the co-stimulatory molecule 4-1BBL would convey potent anti-tumor effects.

IT injections of MVA encoding the TAA ovalbumin (herein referred to as MVA-OVA) controlled tumor growth and prolonged survival of mice bearing established B16.OVA melanomas (FIGS. 1A and 1B). Notably, IT administration of MVA-OVA-4-1BBL increased tumor rejection to 50% of B16.OVA tumor-bearing mice (FIG. 1B). Analysis of peripheral blood lymphocytes (PBLs) after the last IT injection revealed that systemic expansion of TAA-specific CD8⁺ T cells triggered by local MVA-OVA treatment was increased by MVA-OVA-4-1BBL IT administration (FIG. 2A).

Next, we evaluated other independent, established tumor models. IT administration of MVA encoding the endogenous retroviral antigen Gp70 [29 30] (herein referred to as MVA-Gp70) resulted in anti-tumor effects in B16.F10 melanomas (FIGS. 1C and 1D). Interestingly, IT MVA-Gp70-4-1BBL markedly prolonged tumor growth control and significantly improved mouse survival (FIGS. 1C and 1D). Similar results were observed in CT26.WT tumor bearing mice after IT immunization with either MVA-Gp70 or MVA-Gp70-4-1BBL (FIGS. 1E and 1F). Local administration of MVA-Gp70-4-1BBL resulted in over 80% rejection of CT26.WT tumors. Restimulation of PBLs with Gp70-derived peptides revealed a robust induction of interferon gamma (IFN-γ) by p15E (H-2K^{b}-restricted Gp70 peptide)- and AH1 (H-2K^{d}-restricted Gp70 peptide)-specific CD8⁺ T cells upon MVA-Gp70-4-1BBL IT regime in B16.F10 and CT26.WT tumor bearing mice, respectively (FIGS. 2B and 2C, respectively). Of note, over 70% of C57BL/6 mice that were cured upon MVA-OVA-4-1BBL or MVA-Gp70-4-1BBL IT treatment developed vitiligo (FIGS. 2D and 2E). Taken together, IT administration of 4-1BBL adjuvanted MVA potentiates MVA-mediated anti-tumor immune responses, irrespective of the type of tumor antigen or tumor model used.

### Example 3: IT Injected MVA Localizes to the Tumor and Induces Changes in the Tumor Microenvironment (TME)

Having established that T cells rapidly expanded in the TdLN after IT MVA injection raised the question whether replication-deficient MVA would reside exclusively at the site of injection or transit to other organs. To address this, we determined the presence of MVA-derived genomic DNA (gDNA) in tumor, TdLN, NdLN, spleen and blood 6 hours after IT injection. Importantly, MVA gDNA was detected at high amounts in B16.F10 tumors with minimal appearance in the TdLN (FIG. 3A).

We demonstrated that after IT application MVA infection is primarily constrained to the tumor and thus virus-induced anti-tumor immune responses most likely originated in the tumor. Therefore, we hypothesized that IT injection of MVA-TAA-4-1BBL might induce changes in the TME. IT injection of B16.OVA tumors either with MVA or MVA-OVA led to an upregulation of the proinflammatory molecules IFNγ and TNFα compared to PBS. This effect was significantly increased by MVA-OVA-4-1BBL (FIG. 3B). Interestingly, cytokines such as IFNγ and GM-CSF were almost exclusively induced by 4-1BBL adjuvanted MVA (FIG. 3B).

Replicating viruses induce death of infected tumor cells and immune cells [33-35]. Infection with MVA, MVA-TAA or MVA-TAA-4-1BBL enhanced B16.OVA and CT26.WT tumor cell death in vitro (FIG. 7A). Macrophages which have been shown to be preferentially infected by MVA [36] were effectively killed, whereby this effect was significantly increased by 4-1BBL adjuvant (FIG. 7A). Cell death results in the release of intracellular proteins such as High Molecular Group Box 1 (HMGB1) that are sensed by innate immune cells and contribute to the initiation of immune responses [37, 38]. A significant increase of HMGB1 was detected after MVA infection of tumor cells or macrophages irrespective of 4-1BBL (FIG. 7B).

Our results suggest that local injection of MVA resulted in IT expression of MVA encoded genes, leading to the induction of inflammation, cell death of infected cells and release of immunogenic mediators in the TME.

### Example 4: Local MVA Immunotherapy Controls Tumor Growth of Distant Untreated Lesions

The aim of tumor-directed immunotherapy is to generate a systemic anti-tumor immune response that also eradicates distant metastases. As local treatment with MVA-TAA-4-1BBL not only induced robust tumor-specific T cell responses in the TME but also in the blood, we next assessed the systemic anti-tumor potential of IT MVA immunotherapy on distant tumor deposits. CT26.WT tumor cells were implanted subcutaneously to the right and the left flank of Balb/c mice (FIG. 4A). IT injection of MVA-Gp70 delayed tumor growth as compared to PBS (FIGS. 4B and 4C). IT MVA-Gp70-4-1BBL injection resulted in clearance of the treated tumor in 7/10 CT26.WT tumor bearing mice (FIG. 4D). Importantly, local administration of both MVA-Gp70 and MVA-Gp70-4-1BBL led to tumor growth delay and in some cases complete tumor clearance of the untreated tumor lesions (FIGS. 4C and 4D). This data demonstrates the effective induction of anti-tumor immune responses against distant, untreated tumor lesions by IT MVA-Gp70-4-1BBL immunotherapy.

### Example 5: IT MVA-TAA-4-1BBL Treatment Confers Protection from Local and Systemic Tumor Rechallenge

One of the main goals of cancer vaccines is to achieve long-term protective immunological memory to prevent tumor recurrence. Therefore, we first assessed whether IT MVA-TAA-4-1BBL-induced anti-tumor responses generate immunological memory that protects against local tumor rechallenge (FIG. 5A). Naïve mice used as controls rapidly grew tumors, whereas mice that were previously cured after IT MVA-OVA treatment had a high prevalence for tumor regrowth upon rechallenge. In contrast, 80% of mice that previously received IT MVA-OVA-4-1BBL were resistant to secondary tumor growth (9/11) (FIG. 5B). Similar results were obtained in mice that were cured after MVA-Gp70-4-1BBL treatment and local rechallenge with B16.F10 cells. 60% of pre-treated mice remained tumor-free after B16.F10 tumor cell implantation (FIG. 6A). Hence, IT MVA-TAA-4-1BBL treatment induced strong protective immunological memory against local tumor rechallenge.

OVA-specific CD8⁺ T cells could be readily detected prior to rechallenge in mice that had rejected the tumor after IT treatment with MVA-OVA-4-1BBL, but not with MVA-OVA (FIG. 5C). Seven days after tumor cell injection, the OVA-specific T cell population was significantly expanded, indicative of effective tumor recognition (FIG. 5C). Splenocyte OVA₂₅₇₋₂₆₄ peptide restimulation showed that IT MVA-OVA-4-1BBL therapy induced a large population of multi-cytokine-producing antigen-specific CD8⁺ T cells (FIG. 5E). Analysis of spleen, blood, TdLN and NdLN on day 41 after tumor rechallenge revealed an accumulation of OVA-specific CD8⁺ T cells in all organs analysed (FIG. 5D). Memory subset examination [31] revealed that OVA-specific T_{CM} cells were equally distributed over all organs, while T_{EM} cells were mainly found in blood, spleen and TdLN but not in the NdLN (FIG. 5F). Next, we analysed tissue resident memory T cells (T_{RM}) that have been shown to play a key role in anti-tumor immunity [32,33]. Strikingly, we could also detect a significant population of resident memory T cells (T_{RM}) exclusively located in the TdLN (FIG. 5F) [34]. Likewise, p15E-specific CD8⁺ T cells in the blood were detected pre and post rechallenge of mice that cleared primary B16.F10 tumors upon IT MVA-Gp70-4-1BBL treatment (FIG. 6B). Furthermore, we could identify p15E-specific T_{CM}, T_{EM} and T_{RM} cells at day 42 post B16.F10 rechallenge (FIG. 6C-6E), whereby the latter was exclusively found in the TdLN (FIG. 6E). Our results demonstrate that IT MVA-TAA-4-1BBL immunotherapy led to the induction of a diversified population of tumor-specific memory CD8⁺ T cells encompassing T_{CM}, T_{EM} as well as T_{RM} cells.

IT MVA-TAA-4-1BBL injection induced systemic immune responses that mediate control of local recurrent tumors and untreated lesions. We reasoned that the tumor-specific T cell memory generated by IT MVA injection might also protect against metastatic recurrences (FIG. 5G). Macroscopic quantification of tumor nodules in the lung after IV CT26.WT tumor cell injection showed the development of multiple lesions in naïve mice. No macroscopic metastatic lesions were found in the lungs of mice that were previously cured with IT MVA-Gp70 or MVA-Gp70-4-1BBL (FIG. 5H). T cell analysis revealed a population of multifunctional AH1-specific CD8⁺ T cells in MVA-Gp70-4-1BBL cured mice (FIG. 5I).

Taken together, local immunotherapy using MVA genetically modified to express a tumor antigen together with the co-stimulatory molecule 4-1BBL conveyed strong anti-tumor activity by combining innate and adaptive immune activation. This not only resulted in the induction of systemic anti-tumor effects but also in the generation of a potent tumor-specific memory response that protected against local and systemic tumor rechallenge.

### Example 6: Further Discussion of Results

In the present study, we took a novel approach for tumor-directed virotherapy and used a non-replicating MVA genetically modified to express TAAs and the costimulatory molecule 4-1BBL. This combines the excellent immune-stimulatory properties of MVA [12] and its high safety profile [35] with the immune-activating potential of 4-1BBL [22]. IT MVA-TAA-4-1BBL injection activates a sequence of immediate and long-term immune events, ultimately resulting in tumor eradication. The induction of multiple proinflammatory mediators by IT MVA treatment is indicative of a fundamental alteration of the previously immune-suppressive TME that facilitates the reactivation and expansion of tumor specific T cells. MVA-encoded 4-1BBL triggered drastic qualitative and quantitative changes in cytotoxic anti-tumor immune responses that were essential for both, therapeutic efficacy and formation of local and systemic long-term immunologic memory against the primary tumor.

We show for the first time that IT injection of non-replicating MVA conveys potent therapeutic anti-tumor effects. Interestingly, it has been reported that IT delivery of heat inactivated MVA but not MVA induced strong anti-tumor effects mainly depending on the activation of cytotoxic T cells [25]. In contrast to this study, we utilized active MVA encoding for TAA with and without additionally expressing 4-1BBL. Importantly, MVA-TAA alone was already effective in delaying tumor growth and the adjuvantation with 4-1BBL significantly improved therapeutic efficacy, leading to rejection of established tumors within multiple models. Moreover, the anti-tumor effect was independent of the choice of tumor antigen. Apart from the model antigen OVA, we investigated the endogenous retroviral protein Gp70 for its immunogenic potential as TAA. Endogenous retroviral elements are epigenetically silenced in healthy tissues but re-activated and expressed in various cancers [36]. Likewise, Gp70 is highly expressed in several murine tumor cell lines [37]. In humans, there is growing evidence that these non-coding regions might have potent immunogenic properties and therefore represent excellent TAA targets for cancer immunotherapy [38-40]. Given the self-nature of Gp70 [41], the strong therapeutic effects obtained by IT MVA-Gp70-4-1BBL treatment in Gp70-expressing tumor models implies that local MVA therapy cannot only induce the rejection of tumors expressing neoantigens but also break tolerance to endogenous self-antigens.

IT virotherapy repurposes virus-induced inflammation and cell death to alter the immunosuppressive TME [50]. This cascade of events would enhance antitumor-specific immunity. Likewise, our data show that MVA infection promotes tumor cell death and hence HMGB1 release (FIG. 7A, 7B), similar to oncolytic vaccinia virus [51]. Moreover, IT injection of MVA elicited a strong inflammatory response within the TME which was accompanied by the induction of multiple MVA-related cytokines and chemokines [52]. IT application of 4-1BBL-adjuvanted MVA strongly increased the concentration of IFNγ and GM-CSF in B16.OVA tumors (FIG. 3B). This indicates that the induction of those molecules is downstream of 4-1BB signaling. Indeed, in vitro activation of OVA-specific CD8⁺ T cells by MVA-infected tumor cells led to the production of large amounts of IFNγ and GM-CSF exclusively in the presence of MVA-encoded 4-1BBL (FIG. 3B). We investigated MVA-encoded antigen distribution and potential T cell priming in the TdLN and other organs upon IT rMVA administration. We addressed this by performing a comprehensive analysis of the localization of rMVA within different organs after IT injection. MVA gDNA was mostly confined to the tumor site. However, MVA gDNA were also detected in the TdLN, albeit at significantly lower amounts compared to the tumor. Hence, the TdLN could also serve as a priming site for tumor-specific T cells. Our results are in concordance with previous work showing that MVA localizes in the paracortical region of the draining LN after footpad injection of MVA [42]. In agreement to this, no protein or gDNA was found in the NdLN.

An important aspect of tumor-directed immunotherapy is the generation of a systemic anti-tumor immune response that eradicates distant metastases and induces long-term tumor immunity. We showed that local MVA-TAA-4-1BBL treatment not only elicited immune responses within the TME, but also led to systemic antigen-specific CD8⁺ T cell responses in the blood. Moreover, across many individual experiments and different TAAs employed, mice that had rejected melanomas upon IT MVA-TAA-4-1BBL treatment developed vitiligo. Vitiligo is caused by autoreactive CD8 T cells that target the hair pigmenting melanocytes in the skin. These cells have been activated by IT MVA-TAA-4-1BBL virotherapy most likely through antigen spread, a phenomenon that describes the diversification of epitope specificity from the initial focused, dominant epitope-specific immune response, e.g. Gp70 or OVA. Tumor antigen spread is a desirable feature of cancer immunotherapy because it broadens the anti-tumor response and prevents the likelihood of tumor escape by TAA loss.

In addition, our data obtained from the bilateral tumor model unambiguously demonstrated that IT MVA-TAA-4-1BBL injection resulted in significant anti-tumor effects in untreated lesions. These results therefore strongly indicate that the anti-tumor response triggered by local MVA-TAA-4-1BBL administration was associated with system-wide immunity against the primary tumor.

The ability of the immune system to maintain memory of previous antigen encounters is the basis for long-term immunity. Here, we defined the components of immunological memory induced upon IT MVA administration. Circulating TAA-specific CD8⁺ T cells were detected in mice several months after tumor clearance regardless of the tumor model or mouse strain used. CD8⁺ T cell frequencies were significantly increased when 4-1BBL-adjuvanted MVA was used. We found that mice that were previously cured with IT MVA-TAA-4-1BBL were more resistant to subcutaneous tumor rechallenge with B16.OVA or B16.F10 than MVA-TAA treated counterparts. Analysis of tissues from those mice showed that T cell memory subsets were not only found in the circulation but in multiple anatomical sites, suggesting immune surveillance. Increased frequencies of antigen-specific CD8⁺ T_{CM} and T_{EM} subsets were detected in spleen and blood after local tumor rechallenge of cured mice that previously received MVA encoding 4-1BBL. It is well established that 4-1BBL/4-1BB signals are particularly potent in enhancing the expansion and maintenance of CD8 effector and memory T cells [43-45]. Likewise, MVA-encoded 4-1BBL costimulation enhanced the activation and effector function of tumor-specific cytotoxic T cells which resulted in the formation of a potent and diverse memory compartment.

In addition to circulating CD8⁺ T_{CM} and T_{EM} subsets, resident CD8⁺ T_{RM} cells have been shown to cooperate in anti-tumor immunity [32, 33, 46]. Interestingly, cured mice after IT 4-1BBL-adjuvanted MVA showed increased frequencies of tumor-specific T_{RM} cells exclusively in the TdLN after local rechallenge either with B16.OVA or B16.F10. Although CD8⁺ T_{RM} cells were first identified in the tissues, they can also migrate from the tissues and accumulate in the draining LN of mice upon antigen reencounter [34, 46, 47]. In line with our results, 4-1BB has been shown to promote the establishment of an influenza-specific CD8⁺ T_{RM} pool in the lung upon intranasal immunization [48]. We postulate a relationship between the expansion of tumor-specific CD8⁺ T_{RM} cells in the TdLN and the better response to local secondary tumor rechallenge by cured mice upon IT 4-1BBL adjuvanted MVA.

In cancers, memory CD8⁺ T cells are often dysfunctional due to suboptimal differentiation or maintenance conditions and chronic antigen exposure [49]. This phenomenon is associated with the inability to secrete IL-2 and TNFα [50, 51]. Importantly, IT 4-1BBL adjuvanted MVA generated a highly competent CD8⁺ T cell memory pool, that upon reencounter of tumor antigen expanded and produced significant amounts of IFNγ, TNFα and IL-2 compared to IT MVA in all rechallenge models tested.

In summary, we describe a novel therapeutic platform based on the local injection of a non-replicating MVA expressing a tumor antigen in conjunction with 4-1BBL. IT virus injection induced profound proinflammatory changes in the TME leading to reactivation and expansion of tumor-specific CD8⁺ T cells. In addition, we demonstrated the generation of a diverse CD8⁺ T cell memory population protecting from local and systemic tumor rechallenge. Together with the excellent safety profile of MVA, our preclinical data provide a strong rationale for exploring this approach in the clinic.

Final remark: Several documents are cited throughout the text of this specification. Each of the documents cited herein (including all patents, patent applications, scientific publications, manufacturer's specifications, instructions, etc.) are hereby incorporated by reference in their entirety. To the extent, the material incorporated by reference contradicts or is inconsistent with this specification, the specification will supersede any such material. Nothing herein is to be construed as an admission that the invention is not entitled to antedate such disclosure by virtue of prior invention.

### Example 7: MVA Recombinants for Medical Uses

7.1 Construction of MVA Recombinants

Generation of recombinant MVA viruses that embody elements of the present disclosure was done by insertion of the indicated transgenes with their promoters into the vector MVA-BN. Transgenes were inserted using recombination plasmids containing the transgenes and a selection cassette, as well as sequences homologous to the targeted loci within MVA-BN. Homologous recombination between the viral genome and the recombination plasmid was achieved by transfection of the recombination plasmid into MVA-BN infected CEF cells. The selection cassette was then deleted during a second step with help of a plasmid expressing CRE-recombinase, which specifically targets loxP sites flanking the selection cassette, therefore excising the intervening sequence. Alternatively, deletion of the selection cassette was achieved by MVA-mediated recombination using MVA-derived internal repeat sequences.

For generation of recombinant MVA-mBN viruses, CEF cell cultures were each inoculated with MVA-BN and transfected each with the corresponding recombination plasmid. In turn, samples from these cell cultures were inoculated into CEF cultures in medium containing drugs inducing selective pressure, and fluorescence-expressing viral clones were isolated by plaque purification. Loss of the fluorescent-protein-containing selection cassette from these viral clones was mediated in a second step by CRE-mediated recombination involving two loxP sites flanking the selection cassette in each construct or MVA-mediated internal recombination After the second recombination step only the transgene sequences (e.g., 4-1BBL) with their promoters inserted in the targeted loci of MVA-BN were retained. Stocks of plaque-purified virus lacking the selection cassette were prepared. Expression of the identified transgenes is demonstrated in cells inoculated with the described construct.

7.2 Recombinant MVAs Comprising HERV-K Antigens

An MVA-based vector (“MVA-mBN489,” also referred to as “MVA-HERV-Prame-FOLR1-4-1-BBL-CD40L”) was designed comprising TAAs that are proteins of the K superfamily of human endogenous retroviruses (HERV-K), specifically, ERV-K-env and ERV-K-gag. The MVA also was designed to encode human FOLR1 and PRAME, and to express h4-1BBL and hCD40L.

A similar MVA-based vector referred to as “MVA-HERV-Prame-FOLR1-4-1-BBL” was designed to express the TAAs ERV-K-env and ERV-K-gag and human FOLR1 and PRAME, and to express h4-1BBL. Specifically, vector “MVA-BN-4IT” (“MVA-mBN494” or “MVA-HERV-FOLR1-PRAME-h4-1-BBL”) is schematically illustrated in FIG. 8A. HERV-K genes encoding the envelope (env) and group-specific antigen (gag) proteins are usually dormant in healthy human tissue but are activated in many tumors. FOLR1 and PRAME are genes that are specifically upregulated in cells of breast and ovarian cancers. The additional expression of co-stimulatory molecule 4-1-BBL intends to enhance the immune response against the TAAs.

Another MVA-based vector referred to as “MVA-HERV-Prame-FOLR-CD40L was designed to express the TAAs ERV-K-env and ERV-K-gag and human FOLR1 and PRAME, and to express hCD40L. Each of these constructs is useful in methods of the invention.

Exemplary sequences are known in the art and are also set forth in the sequence listing provided. Any sequence can be used in the compositions and methods of the invention so long as it provides the necessary function to the relevant MVA.

For the ERV-K env and gag sequences described above, an amino acid consensus sequence was produced from at least 10 representative sequences, and a potential immunosuppressive domain was inactivated by mutations and replaced in part with the immunodominant T-cell epitope HERV-K-mel as shown below. Suitable sequences are set forth in SEQ ID NO:5 (ERV-K-gag synthetic protein consensus sequence); SEQ ID NO:6 (ERV-K-gag synthetic nucleotide sequence); SEQ ID NO:7 (ERV-K-env/MEL synthetic protein sequence); and SEQ ID NO:8 (ERV-K-env/MEL nucleotide sequence).

MNPSEMQRKAPPRRRRHRNRAPLTHKMNKMVTSEEQMKLPSTKKAEPPTW
AQLKKLTQLATKYLENTKVTQTPESMLLAALMIVSMWVSLPMPAGAAAAN
YTYWAYVPFPPMIRAVTWMDNPIEVYVNDSVWWPGPIDDRCPAKPEEEGM
MINISIGYRYPPICLGRAPGCLMPAVQNWLVEVPTVSPISRFTYHMVSGM
SLRPRVNYLQDFSYQRSLKFRPKGKPCPKEIPKESKNTEVLVWEECVANS
AVILQNNEFGTIIDWAPRGQFYHNCSGQTQSCPSAQVSPAVDSDLTESLD
KHKHKKLQSFYPWEWGEKGISTPRPKIISPVSGPEHPELWRLTVASHHIR
IWSGNQTLETRDRKPFYTVDLNSSLTVPLQSCVKPPYMLVVGNIVIKPDS
QTITCENCRLLTCIDSTFNWQHRILLVRAREGVWIPVSMDRPWEASPSVH
ILTEVLKGVLNRSKRFIFTLIAVIMGLIAVTATAAVAGVALHSSVQSVNF
VNDWQKNSTRLWNSQSSIDQK [symbol] [symbol] QTVIWMGDRLMSLEHRFQL
QCDWNTSDFCITPQIYNESEHHWDMVRRHLQGREDNLTLDISKLKEQIFE
ASKAHLNLVPGTEAIAGVADGLANLNPVTWVKTIGSTTIINLILILVCLF
CLLLVCRCTQQLRRDSDHRERAMMTMAVLSKRKGGNVGKSKRDQIVTVSV

Modified Consensus Amino Acid Sequence of ERVK-Env (Above):
A potential immunosuppressive domain was inactivated by mutations. The introduced mutations replace a substantial portion of the immunosuppressive domain by the immunodominant T-cell epitope HERV-K-mel.

For some of these MVAs, hFOLR1 and PRAME were designed to be produced as a fusion protein. FOLR1 (folate receptor alpha) belongs to the family of folate receptors. It has a high affinity to folic acid and derivatives thereof, and is either secreted or expressed on the cell surface as a membrane protein. The transmembrane protein is anchored to the plasma membrane through a GPI (glycosylphosphatidylinositol) anchor which is most likely attached in the endoplasmic reticulum (ER) through a serine (Ser) residue in the C-terminal region of the protein. To avoid modification of FOLR1 with the GPI-anchor and full processing of the hFOLR1-hPRAME fusion protein in the ER, the C-terminal region from aa 234 to 257 (including the Ser residue) was deleted.

PRAME (Preferentially expressed antigen of melanoma) is a transcriptional regulator protein. It was first described as an antigen in human melanoma, which triggers autologous cytotoxic T cell-mediated immune responses and is expressed in variety of solid and hematological cancers. PRAME inhibits retinoic acid signaling via binding to retinoic acid receptors and thereby might provide a growth advantage to cancer cells. Functionality of PRAME requires nuclear localization, so potential nuclear localization signals (NLS) in PRAME were modified by targeted mutations in the hFOLR1-hPRAME fusion protein.

Thus, for the amino acid sequence of the hFOLR1-hPRAME fusion protein, FOLR1 was modified by deleting the C-terminal GPI anchor signal, while in PRAME, two potential nuclear localization signals were inactivated by amino acid substitutions. In this fusion protein, the N-terminal signal sequence of hFOLR1 should result in ER-targeting and incomplete processing of the fusion protein to serve as an additional safeguard to avoid nuclear localization of PRAME.

The protein sequences of human FOLR1 and human PRAME were based on NCBI RefSeq NP_000793.1 and NP_001278644.1, respectively. In addition to the modifications described above, the nucleotide sequence of the fusion protein was optimized for human codon usage, and poly-nt stretches, repetitive elements, and negative cis-acting elements were removed and the nucleotide sequence is set forth in SEQ ID NO:10 (“hFOLR1Δ_hPRAMEΔ fusion” nucleotide sequence), while the fusion protein sequence is set forth in SEQ ID NO:9.

MAQRMTTQLLLLLVWAVVGEAQTRIAWARTELLNVCMNAKHHKEKPGPED
KLHEQCRPWRKNACCSTNTSQEAHKDVSYLYRFNWNHCGEMAPACKRHFI
QDTCLYECSPNLGPWIQQVDQSWRKERVLNVPLCKEDCEQWWEDCRTSYT
CKSNWHKGWNWTSGFNKCAVGAACQPFHFYFPTPTVLCNEIWTHSYKVSN
YSRGSGRCIQMWFDPAQGNPNEEVARFYAAAMS [symbol] [symbol]
ERRRLWGSIQSRYISMSVWTSPRRLVELAGQSLLKDEA
LAIAALELLPRELFPPLFMAAFDGRHSQTLKAMVQAWPFTCLPLGVLMKG
QHLHLETFKAVLDGLDVLLAQEVRPRRWKLQVLDLRKNSHQDFWTVWSGN
RASLYSFPEPEAAQPMTTKAKVDGLSTEAEQPFIPVEVLVDLFLKEGACD
ELFSYLIEKVAAKKNVLRLCCKKLKIFAMPMQDIKMILKMVQLDSIEDLE
VTCTWKLPTLAKFSPYLGQMINLRRLLLSHIHASSYISPEKEEQYIAQFT
SQFLSLQCLQALYVDSLFFLRGRLDQLLRHVMNPLETLSITNCRLSEGDV
MHLSQSPSVSQLSVLSLSGVMLTDVSPEPLQALLERASATLQDLVFDECG
ITDDQLLALLPSLSHCSQLTTLSFYGNSISISALQSLLQHLIGLSNLTHV
LYPVPLESYEDIHGTLHLERLAYLHARLRELLCELGRPSMVWLSANPCPH
CGDRTFYDPEPILCPCFMPN

Sequence of the hFOLR1-hPRAME Fusion Protein (Above):
Amino acid sequence of the hFOLR1-hPRAME fusion protein, a fusion of modified human FOLR1 (N-terminal portion) and PRAME (C-terminal portion). FOLR1 was modified by deleting the C-terminal GPI anchor signal (strikethrough letters). In PRAME (underlined letters), the initial Methionine was deleted, and two potential nuclear localization signals were inactivated by amino acid substitutions (bold, underlined letters).

The protein sequence of the membrane-bound human 4-1BBL used in this MVA shows 100% identity to NCBI RefSeq NP_003802.1, and the protein sequence of the membrane-bound human CD40L used shows 100% identity to NCBI RefSeq NP_000065.1. For both 4-1BBL and CD40L, the nucleotide sequence was optimized for human codon usage, and poly-nt stretches, repetitive elements, and negative cis-acting elements were removed.

The hCD40L amino acid sequence from NCBI RefSeq NP_000065.1. is set forth in SEQ ID NO:1, while the nucleotide sequence of hCD40L is set forth in SEQ ID NO:2. The h4-1BBL amino acid sequence from NCBI RefSeq NP_003802.1 is set forth in SEQ ID NO:3, while the nucleotide sequence of h4-1BBL is set forth in SEQ ID NO:4.

Each coding region was placed under the control of a different promoter, except that ERV-K-gag and h4-1BBL were both placed under the control of the Pr1328 promoter. The Pr1328 promoter (100 bp in length) is an exact homologue of the Vaccinia Virus Promoter PrB2R. It drives strong immediate early expression as well as late expression at a lower level. In the recombinant MVA-mBN489, the Pr13.5 long promoter drives expression of ERVK-env/MEL. This promoter compromises 124 bp of the intergenic region between 014L/13.5L driving the expression of the native MVA13.5L gene and exhibits a very strong early expression caused by two early promoter core sequences (see Wennier et al. (2013) PLoS One 8(8): e73511). The MVA1-40k promoter, used here to drive expression of hCD40L, was originally isolated as a 161 bp fragment from the vaccinia virus Wyeth Hind III H region in 1986. It compromises 158 bp of the Vaccinia Virus Wyeth and MVA genome within the intergenic region of 094L/095R driving the late gene transcription factor VLTF-4. The promoter PrH5m, used here to drive expression of the hFOLR1-hPRAME fusion protein, is a modified version of the Vaccinia virus H5 gene promoter. It consists of strong early and late elements resulting in expression during both early and late phases of infection of the recombinant MVA (see Wyatt et al. (1996) Vaccine 14: 1451-58).

Based on MVA-mBN494 (see above) still another vector was designed to contain a modification in ERVK-env/MEL. The resulting vector was referred to as “MVA-mBN502” and is schematically illustrated in FIG. 9C. In addition to the modified ERVK-env/MEL, MVA-mBN502 also encodes ERVK-gag, the hFOLR1-hPRAME fusion protein, as well as h4-1BBL.

Natively, HERV-K-env consists of a signal peptide, which is cleaved off post-translationally, a surface (SU) and a transmembrane unit (TM). Cleavage into the two domains is achieved by cellular proteases. An RSKR cleavage motif is required and sufficient for cleavage of the full-length 90 kDa protein into SU (ca. 60 kDa) and TM (ca. 40 kDa) domains. As described above for the preparation of MVA-mBN494, an amino acid consensus sequence for env derived from at least ten representative sequences was generated, and a potential immunosuppressive domain in the TM was inactivated by mutations. The introduced mutations replaced a substantial portion of the immunosuppressive domain by the immunodominant T-cell epitope HERV-K-mel. This transgene (used in MVA-mBN494) was termed ERVK-env/MEL (FIG. 9A).

As compared to MVA-mBN494, the TM domain in ERVK-env/MEL is deleted in MVA-mBN502. This ERVK-env/MEL variant was designated “ERVK-env/MEL_03” and consists of the entire SU domain except for the RSKR furin cleavage site, which was deleted. The MEL peptide was inserted at the C-terminal end, followed by 6 amino acids of the TM domain (excluding the fusion peptide sequence, which is strongly hydrophobic). In addition, this modified ERVK-env/MEL was targeted to the plasma membrane by adding a membrane anchor derived from the human PDGF (platelet-derived growth factor) receptor. This membrane anchor was attached to the SU domain via a flexible glycine-containing linker (FIG. 9B). The resulting ERVK-env/MEL variant, i.e. ERVK-env/MEL_03, is contained in MVA-mBN502 (FIG. 9C). Suitable sequences of the variant are set forth in SEQ ID NO:11 (ERV-K-env/MEL_03 synthetic protein sequence) and SEQ ID NO:12 (ERV-K-env/MEL_03 nucleotide sequence).

7.3 Bioactivity of MVA-HERV-FOLR1-PRAME-h4-1-BBL (MVA-BN-417)

It was investigated whether infection with MVA-BN-4IT (i.e., MVA-HERV-FOLR1-PRAME-h4-1-BBL; see also above) would result in the presentation of vaccine-derived tumor antigens by HLA molecules on human cells. To this end, HLA-ABC peptide complexes on antigen presenting cells were immunoprecipitated, and it was analyzed which HLA-bound peptides could be identified by mass spectrometry.

First, the human monocytic cell line THP-1 was differentiated into macrophages (Daigneault et al. PLoS One, 2010), which exert antigen presenting capabilities, since antigens can be loaded to HLA class I (Nyambura L. et al. J. Immunol 2016). Indeed, THP-1 cells express HLA-A*0201+ which is one of the most frequent haplotypes in the USA and Europe (approximately 30% of the population). Apart of HLA-A*02:01:01G, THP-1 cells were reported to express HLA-B*15 and HLA-C*03 (Battle R. et al., Int. J. of Cancer). Here, 8×105/ml THP-1 cells were cultured in the presence of 200 ng/ml PMA (phorbol-12-myristate-13-acetate) for 3 days before medium was exchanged and cells were cultured for additional 2 days in the absence of PMA. On day 5 cells were infected with MVA-BN-4IT with an InfU (infectious unit) of 4 for 12 hours. As shown in FIG. 8B, HERVK-env/MEL, HERVK-gag and the fusion protein FOLR1-PRAME were expressed after infection of THP-1 cells with MVA-BN-4IT (“mBN494” in FIG. 8B). In contrast, the antigens were not endogenously expressed in uninfected THP-1 cells (“ctr” in FIG. 8B).

Next, a “ProPresent” HLA-ABC ligandome analysis (ProImmune) was performed. In MVA-BN-4IT infected cells, four tumor antigen-derived peptides were identified: The HERV-K env peptide ILTEVLKGV, the HERV-K gag peptide YLSFIKILL and the PRAME peptides ALQSLLQHL and SLLQHLIGL. The two identified PRAME peptides are largely overlapping and most likely share a common core epitope. Both peptides are predicted to bind very strongly to HLA-A*02:01, whereby ALQSLLQHL has almost a similar binding rank to HLA-B*15. Notably, the PRAME peptide SLLQHLIGL has already been described as an immunogenic HLA-A*0201-presented cytotoxic T lymphocyte epitope in human (Kessler J H. et al., J Exp Med., 2001). Altogether, the data demonstrate that the antigens expressed by MVA-BN-4IT can be loaded into HLA of infected cells.

Furthermore, MVA-BN-4IT was tested for its capability of expressing 4-1-BBL in a functional form that binds to its receptor, 4-1-BB. For that purpose, a commercial kit (“4-1BB Bioassay”, Promega) was used. The assay consists of a genetically engineered Jurkat T cell line expressing h4-1-BB and a luciferase reporter driven by a response element (RE) that can respond to 4-1-BB ligand stimulation. When h4-1-BB is stimulated by h4-1-BBL the RE activates cellular luciferase production within the cell. After cell lysis and addition of “Bio-Glo” reagent (Promega), luminescence is measured using a luminometer and quantified. Briefly, HeLa cells were plated (1×10⁶) and infected (TCID₅₀=2) each with the MVA-based constructs indicated in FIG. 8C, cultured overnight (37° C., 5% CO₂), and then co-cultured with the Jurkat-h4-1-BB cells (ratio of HeLa: Jurkat=4:1) for 6 hours. His-tagged h4-1BBL cross-linked with an Fc was used as a reference (positive control) and luciferase expression by Jurkat-h4-1BB cells cultured with 1 μg/ml of the cross-linked h4-1BBI was set to 1 (FIG. 8C, dotted line). MVA-BN (i.e., not encoding h4-1-BBL) was used as a backbone control. As shown in FIG. 8C, HeLa cells infected with an MVA-based vector expressing h4-1-BBL induced a more than 6-fold higher luciferase production (through the co-cultured Jurkat-h4-1-BB cells) as compared to the reference. Notably, luciferase production mediated by MVA-BN-4IT was even higher than that mediated by the other two h4-1-BBL expressing MVA vectors. Thus, MVA-mBN494 expresses functional h4-1-BBL that effectively binds to its 4-1BB receptor.

Sequence listing

The nucleic and amino acid sequences listed in the accompanying sequence
listing are shown using standard letter abbreviations for nucleotide bases, and either one
letter code or three letter code for amino acids, as defined in 37 C.F.R. 1.822. Only one
strand of each nucleic acid sequence is shown, but the complementary strand is
understood as included by any reference to the displayed strand.
Sequences in sequence listing:
SEQ ID NO: 1: hCD40L amino acid sequence from NCBI RefSeq NP_000065.1. (261
amino acids)
SEQ ID NO: 2: hCD40L from NCBI RefSeq NP_000065.1 (792 nucleotides)
SEQ ID NO: 3: h4-1BBL from NCBI RefSeq NP_003802.1 (254 amino acids)
SEQ ID NO: 4: h4-1BBL from NCBI RefSeq NP_003802.1
SEQ ID NO: 5: ERV-K-gag (666 amino acids) synthetic consensus sequence
SEQ ID NO: 6: ERV-K-gag; nt sequence
SEQ ID NO: 7: ERV-K-env/MEL (699 amino acids) synthetic sequence
SEQ ID NO: 8: ERV-K-env/MEL nt sequence
SEQ ID NO: 9: hFOLRIΔ_hPRAMEΔ fusion (741 amino acids)
SEQ ID NO: 10: hFOLRIΔ_hPRAMEΔ fusion (741 amino acids) nt sequence
SEQ ID NO: 11: ERV-K-env/MEL_03 (517 amino acids) synthetic sequence
SEQ ID NO: 12: ERV-K-env/MEL_03 nt sequence
SEQ ID NO: 1
hCD40L from NCBI RefSeq NP_000065.1. (261 amino acids)
MIETYNQTSPRSAATGLPISMKIFMYLLTVFLITQMIGSALFAVYLHRRLDKIEDERNLHE
DFVFMKTIQRCNTGERSLSLLNCEEIKSQFEGFVKDIMLNKEETKKENSFEMQKGDQN
PQIAAHVISEASSKTTSVLQWAEKGYYTMSNNLVTLENGKQLTVKRQGLYYIYAQVTFC
SNREASSQAPFIASLCLKSPGRFERILLRAANTHSSAKPCGQQSIHLGGVFELQPGASV
FVNVTDPSQVSHGTGFTSFGLLKL
SEQ ID NO: 2
hCD40L from NCBI RefSeq NP_000065.1. (792 nucleotides)
nt-Sequence:
atgatcgagacatacaaccagacaagccctagaagcgccgccacaggactgcctatcagcatgaagatcttcatgtacc
tgctgaccgtgttcctgatcacccagatgatcggcagcgccctgtttgccgtgtacctgcacagacggctggacaagatcg
aggacgagagaaacctgcacgaggacttcgtgttcatgaagaccatccagcggtgcaacaccggcgagagaagtctg
agcctgctgaactgcgaggaaatcaagagccagttcgagggcttcgtgaaggacatcatgctgaacaaagaggaaac
qaagaaagagaactccttcgagatgcagaagggcgaccagaatcctcagatcgccgctcacgtgatcagcgagqcca
gcagcaagacaacaagcgtgctgcagtgggccgagaagggctactacaccatgagcaacaacctggtcaccctgga
gaacggcaagcagctgacagtgaagcggcagggcctgtactacatctacgcccaagtgaccttctgcagcaacagag
aggccagctctcaggctcctttcatcgccagcctgtgcctgaagtctcctggcagattcgagcggattctgctgagagccgc
caacacacacagcagcgccaaaccttgtggccagcagtctattcacctcggcggagtgtttgagctgcagcctggcgca
agcgtgttcgtgaatgtgacagaccctagccaggtgtcccacggcaccggctttacatctttcggactgctgaagctgtgat
gatag
SEQ ID NO: 3
h4-1 BBL from NCBI RefSeq NP_003802.1. (254 amino acids)
MEYASDASLDPEAPWPPAPRARACRVLPWALVAGLLLLLLLAAACAVFLACPWAVSG
ARASPGSAASPRLREGPELSPDDPAGLLDLRQGMFAQLVAQNVLLIDGPLSWYSDPG
LAGVSLTGGLSYKEDTKELWAKAGVYYVFFQLELRRWAGEGSGSVSLALHLQPLRS
AAGAAALALTVDLPPASSEARNSAFGFQGRLLHLSAGQRLGVHLHTEARARHAWQLT
QGATVLGLFRVTPEIPAGLPSPRSE
SEQ ID NO: 4
h4-1 BBL from NCBI RefSeq NP_003802.1.
nt sequence:
atggaatacgccagcgacgcctctctggaccctgaagctccttggcctccagctcctagagccagggcttgtagagtgctg
ccttgggctcttgtggctggacttctgcttctgttgctcctggctgctgcctgcgcagtgtttcttgcttgtccatgggctgtgtcagg
agccagagcatctcctggatctgccgcttctcccagactgagagagggacctgaactgagccctgatgatcctgctggact
gctcgacctgagacagggcatgtttgcccagctggtggcccagaatgtgctgctgattgatggccctctgagctggtacag
cgatcctggacttgctggcgttagcctgactggaggcctgagctacaaggaggacaccaaagaactggtggtggccaag
gctggcgtgtactacgtgttctttcagctggaactgcggagagtggtggcaggcgaaggatctggatccgtgtctctggcac
tgcatctgcagcctctgagatctgctgctggtgcagctgccctggctctgacagttgatctgcctcctgcctccagcgaagcc
agaaacagcgcctttggcttccaaggcagactgctgcacctgtctgctggccagagactgggagtgcacctccacacag
aagcaagagcaagacacgcctggcagcttacacaaggcgctacagtgctgggcctgttcagagtgacacctgagattc
cagctggcttgccatctcctcgcagcgagtaatga
SEQ ID NO: 5
ERV-K-env/MEL (699 amino acids)
MNPSEMQRKAPPRRRRHRNRAPLTHKMNKMVTSEEQMKLPSTKKAEPPTWAQLKKL
TQLATKYLENTKVTQTPESMLLAALMIVSMVVSLPMPAGAAAANYTYWAYVPFPPMIR
AVTWMDNPIEVYVNDSVWVPGPIDDRCPAKPEEEGMMINISIGYRYPPICLGRAPGCL
MPAVQNWLVEVPTVSPISRFTYHMVSGMSLRPRVNYLQDFSYQRSLKFRPKGKPCPK
EIPKESKNTEVLVWEECVANSAVILQNNEFGTIIDWAPRGQFYHNCSGQTQSCPSAQV
SPAVDSDLTESLDKHKHKKLQSFYPWEWGEKGISTPRPKIISPVSGPEHPELWRLTVA
SHHIRIWSGNQTLETRDRKPFYTVDLNSSLTVPLQSCVKPPYMLVVGNIVIKPDSQTITC
ENCRLLTCIDSTFNWQHRILLVRAREGVWIPVSMDRPWEASPSVHILTEVLKGVLNRSK
RFIFTLIAVIMGLIAVTATAAVAGVALHSSVQSVNFVNDWQKNSTRLWNSQSSIDQKML
AVISCAVQTVIWMGDRLMSLEHRFQLQCDWNTSDFCITPQIYNESEHHWDMVRRHLQ
GREDNLTLDISKLKEQIFEASKAHLNLVPGTEAIAGVADGLANLNPVTWVKTIGSTTIINLI
LILVCLFCLLLVCRCTQQLRRDSDHRERAMMTMAVLSKRKGGNVGKSKRDQIVTVSV
SEQ ID NO: 6
ERV-K-env/MEL
nt sequence
atgaaccctagcgagatgcagagaaaggctccacctagacggagaagacacagaaacagggctcctctgacacaca
agatgaacaagatggtcaccagcgaggaacagatgaaactgcccagcaccaagaaggccgagcctccaacatggg
ctcagctgaagaaactgacccagctggccaccaagtacctggagaacaccaaagtgacccagacacctgagagcatg
ctgctggcagctctgatgatcgtgtccatggtggtgtccctgcctatgcctgctggtgctgccgctgccaactacacatactgg
gcctacgtgccctttcctcctatgatcaqagccgtgacctggatggacaaccctattgaggtgtacgtgaacgacagcgtgt
gggtgccaggacctatcgacgatagatgtcctgccaaacctgaggaagagggcatgatgatcaacatcagcatcggcta
ccggtatcctccaatctgcctgggcagagcacctggctgtcttatgccagctgtgcagaattggctggtggaagtgcctacc
gtgtctcccatcagccggttcacctaccacatggtgtccggcatgagcctcagacctagagtgaactacttgcaggacttca
gctatcagcggagcctgaagttcagacccaagggaaagccctgtcctaaagagattcccaaagagtccaagaacacc
gaggtgctcgtgtgggaagagtgcgtggccaattctgccgtgatcctgcagaacaacgagttcggcaccatcattgactgg
gctcctagaggccagttctaccacaattgcagcggacagacacagagctgtcctagcgcacaagtgtcaccagccgtgg
atagcgatctgaccgagagcctggacaagcacaaacacaagaaacttcagagcttctatccctgggagtggggagag
aagggcatctctacaccaaggcctaagatcattagccctgtgtctggaccagaacatcccgaactttggagactgacagt
ggccagccaccacatcagaatctggagcggcaatcagaccctggaaacacgggacagaaagcccttctacaccgtcg
atctgaacagcagcctgaccgtgcctctccagagctgtgtgaagcctccttacatgctggtcgtgggcaacattgtgatcaa
gcccgactcccagaccatcacatgcgagaactgcagactgctgacctgcatcgacagcaccttcaactggcagcaccg
gatcctgctcgtgcgagctagagaaggcgtgtggatccctgtctctatggacaggccttgggaagccagccctagcgtgc
acattctgacagaggtgctgaagggcgtgctcaacagatccaagcggttcatcttcaccctgatcgccgtcatcatgggcct
gattgctgtgacagccacagctgctgttgctggcgtggccctgcatagctctgtgcagagcgtgaacttcgtgaacgattgg
cagaagaacagcacacggctgtggaacagccagagcagcatcgaccagaagatgctggccgtgatctcctgtgccgt
gcagacagttatctggatgggcgacagactgatgagcctggaacaccggttccagctgcagtgcgactggaataccagc
gacttctgcatcacacctcagatctacaacgagagcgagcaccactgggatatggtccgaaggcatctgcagggcaga
gaggacaacctgacactggacatcagcaagctgaaagagcagatcttcgaggccagcaaggctcacctgaatctggtg
cctggaaccgaagctattgctggagttgcagatggcctggccaatctgaatcctgtgacctgggtcaagaccatcggcag
caccacaatcatcaacctgatcctgatcctcgtgtgcctgttttgcctgctgcttgtgtgcagatgcacccagcagctgagaa
gagacagcgaccatagagaaagagccatgatgaccatggccgtcctgagcaagagaaagggaggcaacgtgggca
agagcaagcgggatcagatcgtgaccgtgtccgtttgataa
SEQ ID NO: 7
ERV-K-gag (666 amino acids)
MGQTKSKIKSKYASYLSFIKILLKRGGVKVSTKNLIKLFQIIEQFCPWFPEQGTLDLKDW
KRIGKELKQAGRKGNIIPLTVWNDWAIIKAALEPFQTEEDSVSVSDAPGSCIIDCNENTR
KKSQKETESLHCEYVAEPVMAQSTQNVDYNQLQEVIYPETLKLEGKGPELVGPSESKP
RGTSPLPAGQVPVTLQPQKQVKENKTQPPVAYQYWPPAELQYRPPPESQYGYPGMP
PAPQGRAPYPQPPTRRLNPTAPPSRQGSELHEIIDKSRKEGDTEAWQFPVTLEPMPP
GEGAQEGEPPTVEARYKSFSIKMLKDMKEGVKQYGPNSPYMRTLLDSIAHGHRLIPYD
WEILAKSSLSPSQFLQFKTWWIDGVQEQVRRNRAANPPVNIDADQLLGIGQNWSTISQ
QALMQNEAIEQVRAICLRAWEKIQDPGSTCPSFNTVRQGSKEPYPDFVARLQDVAQK
SIADEKARKVIVELMAYENANPECQSAIKPLKGKVPAGSDVISEYVKACDGIGGAMHKA
MLMAQAITGWVLGGQVRTFGGKCYNCGQIGHLKKNCPVLNKQNITIQATTTGREPPDL
CPRCKKGKHWASQCRSKFDKNGQPLSGNEQRGQPQAPQQTGAFPIQPFVPQGFQG
QQPPLSQVFQGISQLPQYNNCPPPQAAVQQ
SEQ ID NO: 8
ERV-K-gag
nt sequence
atgggacagaccaagagtaagatcaagtctaagtacgccagctacctcagcttcatcaagatcctgctgaagagagga
ggcgtgaaagtgtccaccaagaacctgatcaagctgttccagatcatcgagcagttctgtccctggtttcctgagcagggc
accctggatctgaaggactggaagcggatcggcaaagagctgaagcaggctggcagaaagggcaacatcatccctct
gaccgtgtggaacgactgggccatcatcaaagcagctctggaacccttccagaccgaagaggatagcgtgtccgtgtct
gatgctcctggcagctgcatcatcgactgcaacgagaacacccggaagaagtcccagaaagagacagagagcctgc
actgcgagtacgtggccgaacctgtgatggctcagagcacccagaacgtggactacaaccagctccaagaagtgatct
atcccgaaacactgaagctggaaggcaagggacctgaactcgtgggtccttctgagtctaagcccagaggcacatctcc
tctgcctgcaggacaggtgccagtgacactgcagcctcagaaacaagtgaaagagaacaagacccagcctcctgtgg
cctaccagtattggcctccagccgagctgcagtacagacctcctccagagagccagtacggctaccctggaatgcctcct
gctcctcaaggcagagctccttatcctcagcctcctaccagacggctgaaccctacagctcctcctagcagacagggctct
gagctgcacgagatcattgacaagagccggaaagagggcgacaccgaggcttggcagtttcccgttacactggaaccc
atgcctccaggcgaaggcgctcaagaaggcgaacctcctacagtggaagccaggtacaagagcttcagcatcaagat
gctgaaggacatgaaggaaggcgtcaagcagtacggacctaacagcccatacatgcggaccctgctggattctattgcc
cacggccaccggctgatcccttacgattgggagatcctggctaagtcctctctgagccctagccagttcctgcagttcaaga
cctggtggatcgacggcgtgcaagaacaagtgagacggaacagagctgccaatcctcctgtgaacatcgacgccgac
cagctcctcggaatcggccagaattggagcaccatctctcagcaggctctgatgcagaacgaggccattgaacaagtca
gagccatctgcctgagagcttgggagaagattcaggacccaggcagcacatgtcccagcttcaataccgttcggcaggg
cagcaaagagccctatcctgactttgtggctagactgcaggatgtggcccagaagtctattgccgacgagaaggctcgga
aagtgatcgtggaactgatggcctacgagaacgctaatccagagtgccagagcgccatcaagcccttgaagggcaaag
tgcctgccggatccgatgtgatcagcgagtatgtgaaggcctgcgacggaatcggaggtgccatgcacaaagccatgct
gatggcacaggccatcactggcgttgtgctcggaggacaagttcggacctttggaggcaagtgctacaactgtggccaga
tcggacacctgaagaagaactgccctgtgctgaacaagcagaacatcaccatccaggccaccaccaccggcagaga
acctccagatctgtgccctagatgcaagaagggcaagcactgggccagccagtgcagaagcaagttcgacaagaacg
gccagcctctgagcggcaacgaacaaagaggacagcctcaggctcctcagcagactggcgcatttccaatccagccct
tcgtgcctcaaggcttccagggacaacagcctccactgtctcaggtgttccagggcattagccagctccctcagtacaaca
actgccctccacctcaggctgctgtgcagcagtgatga
SEQ ID NO: 9
hFOLRIΔ hPRAMEΔ fusion (741 amino acids)
MAQRMTTQLLLLLVWWAVVGEAQTRIAWARTELLNVCMNAKHHKEKPGPEDKLHEQC
RPWRKNACCSTNTSQEAHKDVSYLYRFNWNHCGEMAPACKRHFIQDTCLYECSPNL
GPWIQQVDQSWRKERVLNVPLCKEDCEQWWEDCRTSYTCKSNWHKGWNWTSGFN
KCAVGAACQPFHFYFPTPTVLCNEIWTHSYKVSNYSRGSGRCIQMWFDPAQGNPNEE
VARFYAAAMERRRLWGSIQSRYISMSVWTSPRRLVELAGQSLLKDEALAIAALELLPRE
LFPPLFMAAFDGRHSQTLKAMVQAWPFTCLPLGVLMKGQHLHLETFKAVLDGLDVLLA
QEVRPRRWKLQVLDLRKNSHQDFWTVWSGNRASLYSFPEPEAAQPMTTKAKVDGLS
TEAEQPFIPVEVLVDLFLKEGACDELFSYLIEKVAAKKNVLRLCCKKLKIFAMPMQDIKMI
LKMVQLDSIEDLEVTCTWKLPTLAKFSPYLGQMINLRRLLLSHIHASSYISPEKEEQYIA
QFTSQFLSLQCLQALYVDSLFFLRGRLDQLLRHVMNPLETLSITNCRLSEGDVMHLSQ
SPSVSQLSVLSLSGVMLTDVSPEPLQALLERASATLQDLVFDECGITDDQLLALLPSLS
HCSQLTTLSFYGNSISISALQSLLQHLIGLSNLTHVLYPVPLESYEDIHGTLHLERLAYLH
ARLRELLCELGRPSMVWLSANPCPHCGDRTFYDPEPILCPCFMPN
SEQ ID NO: 10
hFOLRIΔ hPRAMEΔ fusion (741 amino acids)
nt sequence
tggcccagagaatgaccacacaactgctgctgctcctggtgtgggttgccgttgttggagaggcccagaccagaattgcct
gggccagaaccgagctgctgaacgtgtgcatgaacgccaagcatcacaaagagaagcctggacctgaagacaagct
gcatgaacagtgtcggccttggagaaagaatgcttgctgtagcaccaacaccagccaagaggcccacaaggacgtgtc
ctacctgtaccggttcaactggaaccactgcggagaaatggctcctgcctgcaagagacacttcatccaggatacctgcct
gtacgagtgctctcccaatctcggaccttggatccagcaagtggaccagagctggcggaaagaacgggtgctgaatgtg
cccttgtgcaaagaggattgcgagcagtggtgggaagattgccggaccagctacacatgtaagagcaactggcacaaa
ggctggaactggaccagcggcttcaacaagtgtgccgtgggagctgcctgccagcctttccacttctacttcccaacacct
accgtgctgtgcaacgaaatctggacccacagctacaaggtgtccaactacagcagaggcagcggcaggtgtatccag
atgtggttcgatcccgctcagggcaatcccaatgaggaagtggctagattctacgctgctgccatggaaagaagaaggct
ctggggcagcatccagagccggtacattagcatgagcgtgtggacaagccctagacggctggttgaactggctggacag
agcctgctcaaggatgaggccctggccattgctgctctggagctgctgcctagagagctgttccctcctctgttcatggctgc
cttcgacggcagacacagccagacactgaaagccatggtgcaggcctggectttcacctgtctgcctctgggagtgctgat
gaagggccagcatctgcacctggaaaccttcaaggccgtgctggacggcctggatgttctcctggctcaagaggtgagg
cctcggcgttggaaactgcaggttctggatctgcggaagaactctcaccaggatttctggaccgtttggtccggcaacaga
gccagcctgtacagctttcctgaacctgaggctgcccagcccatgaccacaaaggccaaagtggatggcctgagcaca
gaggccgagcagcctttcattcccgtcgaagtgctggtggacctgttcctgaaagaaggagcctgcgatgagctgttcagc
tacctgattgagaaggtggcagccaagaagaacgtgctgcggctgtgctgcaagaagctgaagatctttgccatgcctat
gcaggatatcaagatgatcctgaagatggtgcagctggacagcatcgaggacctggaagtgacctgtacctggaagctg
cccacactggccaagttcagcccttacctgggacagatgattaacctgcggaggctgctgctgtctcacatccacgccagc
tcctacatcagccctgagaaagaggaacagtatatcgcccagttcacaagccagtttctgagcctgcagtgtctgcaggcc
ctgtacgtggacagcctgttctttctgagaggcaggctggatcagctgctgcggcacgtgatgaaccctctggaaaccctg
agcatcaccaactgtagactgagcgagggcgacgtgatgcacctgtctcagagcccatctgtgtctcagctgagcgtgct
gtctctgtctggcgtgatgctgaccgatgtgagccctgaacctctgcaggcactgctggaaagagcctccgctactctgcag
gacctggtgttcgatgagtgcggcatcaccgatgaccagctgcttgctctgctgccaagcctgagccactgtagccagctg
acaaccctgtccttctacggcaacagcatctccatctctgccctgcagtctctcctgcagcatctgatcggcctgtccaatctg
acccacgtgctgtaccctgtgccactggaaagctacgaggacatccacggaaccctgcacctcgagagactggcctatc
tgcatgctcggctgagagaactgctgtgcgaactgggcagacccagcatggtttggctgagcgccaatccatgtcctcact
gtggcgaccggaccttctacgaccctgagcctatcctgtgtccttgcttcatgcccaactaatag
SEQID NO: 11
ERV-K-env/MEL 03 (517 amino acids)
MNPSEMQRKAPPRRRRHRNRAPLTHKMNKMVTSEEQMKLPSTKKAEPPTWAQLKKL
TQLATKYLENTKVTQTPESMLLAALMIVSMVVSLPMPAGAAAANYTYWAYVPFPPMIR
AVTWMDNPIEVYVNDSVWVPGPIDDRCPAKPEEEGMMINISIGYRYPPICLGRAPGCL
MPAVQNWLVEVPTVSPISRFTYHMVSGMSLRPRVNYLQDFSYQRSLKFRPKGKPCPK
EIPKESKNTEVLVWEECVANSAVILQNNEFGTIIDWAPRGQFYHNCSGQTQSCPSAQV
SPAVDSDLTESLDKHKHKKLQSFYPWEWGEKGISTPRPKIISPVSGPEHPELWRLTVA
SHHIRIWSGNQTLETRDRKPFYTVDLNSSLTVPLQSCVKPPYMLVVGNIVIKPDSQTITC
ENCRLLTCIDSTFNWQHRILLVRAREGVWIPVSMDRPWEASPSVHILTEVLKGVLNMLA
VISCAVAGVALHGSAGSAAGSGEFVVISAILALVVLTIISLIILIMLWQKKPR
SEQ ID NO: 12
ERV-K-env/MEL 03
nt sequence
atgaaccctagcgagatgcagagaaaggctccacctagacggagaagacacagaaacagggctcctctgacacaca
agatgaacaagatggtcaccagcgaggaacagatgaaactgcccagcaccaagaaggccgagcctccaacatggg
ctcagctgaagaaactgacccagctggccaccaagtacctggagaacaccaaagtgacccagacacctgagagcatg
ctgctggcagctctgatgatcgtgtccatggtggtgtccctgcctatgcctgctggtgctgccgctgccaactacacatactgg
gcctacgtgccctttcctcctatgatcagagccgtgacctggatggacaaccctattgaggtgtacgtgaacgacagcgtgt
gggtgccaggacctatcgacgatagatgtcctgccaaacctgaggaagagggcatgatgatcaacatcagcatcggcta
ccggtatcctccaatctgcctgggcagagcacctggctgtcttatgccagctgtgcagaattggctggtggaagtgcctacc
gtgtctcccatcagccggttcacctaccacatggtgtccggcatgagcctcagacctagagtgaactacttgcaggacttca
gctatcagcggagcctgaagttcagacccaagggaaagccctgtcctaaagagattcccaaagagtccaagaacacc
gaggtgctcgtgtgggaagagtgcgtggccaattctgccgtgatcctgcagaacaacgagttcggcaccatcattgactgg
gctcctagaggccagttctaccacaattgcagcggacagacacagagctgtcctagcgcacaagtgtcaccagccgtgg
atagcgatctgaccgagagcctggacaagcacaaacacaagaaacttcagagcttctatccctgggagtggggagag
aagggcatctctacaccaaggcctaagatcattagccctgtgtctggaccagaacatcccgaactttggagactgacagt
ggccagccaccacatcagaatctggagcggcaatcagaccctggaaacacgggacagaaagcccttctacaccgtcg
atctgaacagcagcctgaccgtgcctctccagagctgtgtgaagcctccttacatgctggtcgtgggcaacattgtgatcaa
gcccgactcccagaccatcacatgcgagaactgcagactgctgacctgcatcgacagcaccttcaactggcagcaccg
gatcctgctcgtgcgagctagagaaggcgtgtggatccctgtctctatggacaggccttgggaagccagccctagcgtgc
acattctgacagaggtgctgaagggcgtgctcaacatgctggccgtgatctcctgtgccgtggctggcgtggccctgcatg
gctctgctggatctgctgctggaagcggcgagttcgtggtcatctctgccattctggctctggtggtgctgaccatcatcagcct
gatcatcctgattatgctgtggcagaagaagccccggtgataa

### REFERENCES

1. Pardoll D M. The blockade of immune checkpoints in cancer immunotherapy. Nat Rev Cancer 2012; 12(4):252-64. doi: 10.1038/nrc3239 [published Online First: 2012/03/23]
2. Aznar M A, Tinari N, Rullan A J, et al. Intratumoral Delivery of Immunotherapy-Act Locally, Think Globally. J Immunol 2017; 198(1):31-39. doi: 10.4049/jimmunol.1601145 [published Online First: 2016/12/21]
3. Ellmark P, Mangsbo S M, Furebring C, et al. Tumor-directed immunotherapy can generate tumor-specific T cell responses through localized co-stimulation. Cancer Immunol Immunother 2017; 66(1):1-7. doi: 10.1007/s00262-016-1909-3 [published Online First: 2016/10/08]
4. Pol J, Kroemer G, Galluzzi L. First oncolytic virus approved for melanoma immunotherapy. Oncoimmunology 2016; 5(1):e1115641. doi: 10.1080/2162402X.2015.1115641 [published Online First: 2016/03/05]
5. Kaufman H L, Kohlhapp F J, Zloza A. Oncolytic viruses: a new class of immunotherapy drugs. Nat Rev Drug Discov 2015; 14(9):642-62. doi: 10.1038/nrd4663
6. Davola M E, Mossman K L. Oncolytic viruses: how “lytic” must they be for therapeutic efficacy? Oncoimmunology 2019; 8(6):e1581528. doi: 10.1080/2162402X.2019.1596006 [published Online First: 2019/05/10]
7. Russell S J, Barber G N. Oncolytic Viruses as Antigen-Agnostic Cancer Vaccines. Cancer Cell 2018; 33(4):599-605. doi: 10.1016/j.ccell.2018.03.011
8. Russell S J, Peng K W, Bell J C. Oncolytic virotherapy. Nat Biotechnol 2012; 30(7):658-70. doi: 10.1038/nbt.2287
9. Pittman P R, Hahn M, Lee H S, et al. Phase 3 Efficacy Trial of Modified Vaccinia Ankara as a Vaccine against Smallpox. N Engl J Med 2019; 381(20):1897-908. doi: 10.1056/NEJMoa1817307 [published Online First: 2019/11/14]
10. Anywaine Z, Whitworth H, Kaleebu P, et al. Safety and Immunogenicity of a 2-Dose Heterologous Vaccination Regimen With Ad26.ZEBOV and MVA-BN-Filo Ebola Vaccines: 12-Month Data From a Phase 1 Randomized Clinical Trial in Uganda and Tanzania. J Infect Dis 2019; 220(1):46-56. doi: 10.1093/infdis/jiz070 [published Online First: 2019/02/24]
11. Waibler Z, Anzaghe M, Frenz T, et al. Vaccinia virus-mediated inhibition of type I interferon responses is a multifactorial process involving the soluble type I interferon receptor B18 and intracellular components. J Virol 2009; 83(4):1563-71. doi: 10.1128/JVI.01617-08 [published Online First: 2008/12/17]
12. Price P J, Torres-Dominguez L E, Brandmuller C, et al. Modified Vaccinia virus Ankara: innate immune activation and induction of cellular signalling. Vaccine 2013; 31(39):4231-4. doi: 10.1016/j.vaccine.2013.03.017 [published Online First: 2013/03/26]
13. Samuelsson C, Hausmann J, Lauterbach H, et al. Survival of lethal poxvirus infection in mice depends on TLR9, and therapeutic vaccination provides protection. J Clin Invest 2008; 118(5):1776-84. doi: 10.1172/JCI33940 [published Online First: 2008/04/10]
14. Harrer E, Bauerle M, Ferstl B, et al. Therapeutic vaccination of HIV-1-infected patients on HAART with a recombinant HIV-1 nef-expressing MVA: safety, immunogenicity and influence on viral load during treatment interruption. Antivir Ther 2005; 10(2):285-300. [published Online First: 2005/05/04]
15. Suter M, Meisinger-Henschel C, Tzatzaris M, et al. Modified vaccinia Ankara strains with identical coding sequences actually represent complex mixtures of viruses that determine the biological properties of each strain. Vaccine 2009; 27(52):7442-50. doi: 10.1016/j.vaccine.2009.05.095 [published Online First: 2009/06/23]
16. Lauterbach H, Patzold J, Kassub R, et al. Genetic Adjuvantation of Recombinant MVA with CD40L Potentiates CD8 T Cell Mediated Immunity. Front Immunol 2013; 4:251. doi: 10.3389/fimmu.2013.00251 [published Online First: 2013/08/30]
17. Medina-Echeverz J, Hinterberger M, Testori M, et al. Synergistic cancer immunotherapy combines MVA-CD40L induced innate and adaptive immunity with tumor targeting antibodies. Nat Commun 2019; 10(1):5041. doi: 10.1038/s41467-019-12998-6 [published Online First: 2019/11/07]
18. Lawler S E, Speranza M C, Cho C F, et al. Oncolytic Viruses in Cancer Treatment: A Review. JAMA Oncol 2017; 3(6):841-49. doi: 10.1001/jamaoncol.2016.2064
19. Watts CWGHYLAJMTH. Immune regulation by 4-1BB and 4-1BBL: complexities and challenges. Immunological Reviews 2009
20. Hendriks J, Xiao Y, Rossen J W, et al. During viral infection of the respiratory tract, CD27, 4-1BB, and OX40 collectively determine formation of CD8+ memory T cells and their capacity for secondary expansion. J Immunol 2005; 175(3):1665-76. doi: 10.4049/jimmunol.175.3.1665
21. Palazon A, Martinez-Forero I, Teijeira A, et al. The HIF-1alpha hypoxia response in tumor-infiltrating T lymphocytes induces functional CD137 (4-1BB) for immunotherapy. Cancer Discov 2012; 2(7):608-23. doi: 10.1158/2159-8290.CD-11-0314 [published Online First: 2012/06/22]
22. Bartkowiak T, Curran M A. 4-1BB Agonists: Multi-Potent Potentiators of Tumor Immunity. Front Oncol 2015; 5:117. doi: 10.3389/fonc.2015.00117 [published Online First: 2015/06/25]
23. Imai C, Mihara K, Andreansky M, et al. Chimeric receptors with 4-1BB signaling capacity provoke potent cytotoxicity against acute lymphoblastic leukemia. Leukemia 2004, 18(4):676-84. doi: 10.1038/sj.leu.2403302
24. Nakao S, Arai Y, Tasaki M, et al. Intratumoral expression of IL-7 and IL-12 using an oncolytic virus increases systemic sensitivity to immune checkpoint blockade. Sci Transl Med 2020; 12(526) doi: 10.1126/scitranslmed.aax7992 [published Online First: 2020/01/17]
25. Dai P, Wang W, Yang N, et al. Intratumoral delivery of inactivated modified vaccinia virus Ankara (iMVA) induces systemic anti-tumor immunity via STING and Batf3-dependent dendritic cells. Sci Immunol 2017; 2(11) doi: 10.1126/sciimmunol.aa11713 [published Online First: 2017/08/02]
26. Kudo-Saito C, Schlom J, Hodge J W. Intratumoral vaccination and diversified subcutaneous/intratumoral vaccination with recombinant poxviruses encoding a tumor antigen and multiple costimulatory molecules. Clin Cancer Res 2004, 10(3):1090-9. doi: 10.1158/1078-0432.ccr-03-0145 [published Online First: 2004/02/12]
27. Rivadeneira D B, DePeaux K, Wang Y, et al. Oncolytic Viruses Engineered to Enforce Leptin Expression Reprogram Tumor-Infiltrating T Cell Metabolism and Promote Tumor Clearance. Immunity 2019; 51(3):548-60 e4. doi: 10.1016/j.immuni.2019.07.003 [published Online First: 2019/09/01]
28. Kim H S, Kim-Schulze S, Kim D W, et al. Host lymphodepletion enhances the therapeutic activity of an oncolytic vaccinia virus expressing 4-1BB ligand. Cancer Res 2009, 69(21):8516-25. doi: 10.1158/0008-5472.CAN-09-2522
29. Huang A Y, Gulden P H, Woods A S, et al. The immunodominant major histocompatibility complex class I-restricted antigen of a murine colon tumor derives from an endogenous retroviral gene product. Proc Natl Acad Sci USA 1996; 93(18):9730-5. doi: 10.1073/pnas.93.18.9730 [published Online First: 1996/09/03]
30. Risser R, Horowitz J M, McCubrey J. Endogenous mouse leukemia viruses. Annu Rev Genet 1983; 17:85-121. doi: 10.1146/annurev.ge.17.120183.000505 [published Online First: 1983/01/01]
31. Kaech S M, Tan J T, Wherry E J, et al. Selective expression of the interleukin 7 receptor identifies effector CD8 T cells that give rise to long-lived memory cells. Nat Immunol 2003, 4(12):1191-8. doi: 10.1038/ni1009 [published Online First: 2003/11/20]
32. Enamorado M, Iborra S, Priego E, et al. Enhanced anti-tumour immunity requires the interplay between resident and circulating memory CD8(+) T cells. Nat Commun 2017; 8:16073. doi: 10.1038/ncomms16073 [published Online First: 2017/07/18]
33. Nizard M, Roussel H, Diniz M O, et al. Induction of resident memory T cells enhances the efficacy of cancer vaccine. Nat Commun 2017; 8:15221. doi: 10.1038/ncomms15221 [published Online First: 2017/05/26]
34. Beura L K, Wijeyesinghe S, Thompson E A, et al. T Cells in Nonlymphoid Tissues Give Rise to Lymph-Node-Resident Memory T Cells. Immunity 2018; 48(2):327-38 e5. doi: 10.1016/j.immuni.2018.01.015 [published Online First: 2018/02/22]
35. Overton E T, Lawrence S J, Wagner E, et al. Immunogenicity and safety of three consecutive production lots of the non replicating smallpox vaccine MVA: A randomised, double blind, placebo controlled phase III trial. PLoS One 2018; 13(4):e0195897. doi: 10.1371/journal.pone.0195897 [published Online First: 2018/04/14]
36. Kassiotis G, Stoye J P. Immune responses to endogenous retroelements: taking the bad with the good. Nat Rev Immunol 2016, 16(4):207-19. doi: 10.1038/nri.2016.27
37. Scrimieri F, Askew D, Corn D J, et al. Murine leukemia virus envelope gp70 is a shared biomarker for the high-sensitivity quantification of murine tumor burden. Oncoimmunology 2013; 2(11):e26889. doi: 10.4161/onci.26889
38. Attermann A S, Bjerregaard A M, Saini S K, et al. Human endogenous retroviruses and their implication for immunotherapeutics of cancer. Ann Oncol 2018; 29(11):2183-91. doi: 10.1093/annonc/mdy413
39. Wang-Johanning F, Radvanyi L, Rycaj K, et al. Human endogenous retrovirus K triggers an antigen-specific immune response in breast cancer patients. Cancer Res 2008, 68(14):5869-77. doi: 10.1158/0008-5472.CAN-07-6838
40. Kong Y, Rose C M, Cass A A, et al. Transposable element expression in tumors is associated with immune infiltration and increased antigenicity. Nat Commun 2019; 10(1):5228. doi: 10.1038/s41467-019-13035-2
41. McWilliams J A, Sullivan R T, Jordan K R, et al. Age-dependent tolerance to an endogenous tumor-associated antigen. Vaccine 2008; 26(15):1863-73. doi: 10.1016/j.vaccine.2008.01.052 [published Online First: 2008/03/11]
42. Reynoso G V, Weisberg A S, Shannon J P, et al. Lymph node conduits transport virions for rapid T cell activation. Nat Immunol 2019; 20(5):602-12. doi: 10.1038/s41590-019-0342-0 [published Online First: 2019/03/20]
43. Sanchez-Paulete A R, Labiano S, Rodriguez-Ruiz M E, et al. Deciphering CD137 (4-1BB) signaling in T-cell costimulation for translation into successful cancer immunotherapy. Eur J Immunol 2016; 46(3):513-22. doi: 10.1002/eji.201445388 [published Online First: 2016/01/17]
44. Bertram E M, Lau P, Watts T H. Temporal segregation of 4-1BB versus CD28-mediated costimulation: 4-1BB ligand influences T cell numbers late in the primary response and regulates the size of the T cell memory response following influenza infection. J Immunol 2002; 168(8):3777-85. doi: 10.4049/jimmunol.168.8.3777 [published Online First: 2002/04/09]
45. Willoughby J E, Kerr J P, Rogel A, et al. Differential impact of CD27 and 4-1BB costimulation on effector and memory CD8 T cell generation following peptide immunization. J Immunol 2014; 193(1):244-51. doi: 10.4049/jimmunol.1301217 [published Online First: 2014/05/27]
46. Park S L, Buzzai A, Rautela J, et al. Tissue-resident memory CD8(+) T cells promote melanoma-immune equilibrium in skin. Nature 2019; 565(7739):366-71. doi: 10.1038/s41586-018-0812-9 [published Online First: 2019/01/02]
47. Schenkel J M, Fraser K A, Masopust D. Cutting edge: resident memory CD8 T cells occupy frontline niches in secondary lymphoid organs. J Immunol 2014; 192(7):2961-4. doi: 10.4049/jimmunol.1400003 [published Online First: 2014/03/07]
48. Zhou A C, Wagar L E, Wortzman M E, et al. Intrinsic 4-1BB signals are indispensable for the establishment of an influenza-specific tissue-resident memory CD8 T-cell population in the lung. Mucosal Immunol 2017; 10(5):1294-309. doi: 10.1038/mi.2016.124 [published Online First: 2017/01/05]
49. Reading J L, Galvez-Cancino F, Swanton C, et al. The function and dysfunction of memory CD8(+) T cells in tumor immunity. Immunol Rev 2018; 283(1):194-212. doi: 10.1111/imr.12657 [published Online First: 2018/04/18]
50. Wherry E J, Blattman J N, Murali-Krishna K, et al. Viral persistence alters CD8 T-cell immunodominance and tissue distribution and results in distinct stages of functional impairment. J Virol 2003; 77(8):4911-27. doi: 10.1128/jvi.77.8.4911-4927.2003 [published Online First: 2003/03/29]
51. Appay V, Nixon D F, Donahoe S M, et al. HIV-specific CD8(+) T cells produce antiviral cytokines but are impaired in cytolytic function. J Exp Med 2000; 192(1):63-75. doi: 10.1084/jem.192.1.63 [published Online First: 2000/07/06]

We claim:

1-13. (canceled)

14. A method of stimulating an immune response in a subject having a plurality of tumors, comprising a step of locally administering to fewer than all of the tumors in said subject a recombinant MVA comprising at least one first nucleic acid encoding a TAA and a second nucleic acid encoding 4-1-BBL, wherein an immune response to the TAA is stimulated in the subject.

15. A method of treating a subject having at least one inaccessible tumor and at least one accessible tumor, comprising locally administering to at least one accessible tumor in the subject a recombinant MVA comprising at least one first nucleic acid encoding a TAA and a second nucleic acid encoding 4-1-BBL, whereby the growth of the inaccessible tumor is decreased or stopped.

16. The method of claim 14, wherein said first nucleic acid encodes an endogenous retroviral protein (ERV), carcinoembryonic antigen (CEA), mucin 1 cell surface associated (MUC-1), human epidermal growth factor receptor 2 (HER-2), tyrosine related protein 1 (TRP1), tyrosine related protein 1 (TRP2), Brachyury, folate receptor alpha (FOLR1), preferentially expressed antigen of melanoma (PRAME), or the endogenous retroviral peptide MEL; and combinations thereof.

17. The method of claim 16, wherein the ERV is a HERV-K envelope protein (HERV-K-env), a HERV-K gag protein, or HERV-K-env/MEL.

18. The method of claim 16, wherein said first nucleic acid encodes a fusion protein of FOLR1 and PRAME.

19. The method of claim 15, wherein said first nucleic acid encodes an endogenous retroviral protein (ERV), carcinoembryonic antigen (CEA), mucin 1 cell surface associated (MUC-1), human epidermal growth factor receptor 2 (HER-2), tyrosine related protein 1 (TRP1), tyrosine related protein 1 (TRP2), Brachyury, folate receptor alpha (FOLR1), preferentially expressed antigen of melanoma (PRAME), or the endogenous retroviral peptide MEL; and combinations thereof.

20. The method of claim 19, wherein the ERV is a HERV-K envelope protein (HERV-K-env), a HERV-K gag protein, or HERV-K-env/MEL.

21. The method of claim 19, wherein said first nucleic acid encodes a fusion protein of FOLR1 and PRAME.
